(19) United States
(12) Patent Application Publication
(10) Pub. No.: US 2007/0077221 A1
(43) Pub. Date: Apr. 5, 2007

(54) Cosmetic composition comprising an ampholytic copolymer and another agent

(76) Inventors: Aline Seigneurin, Le Chesnay (FR); Carole Foucault, Charenton Le Pont (FR); Eric Leroy, Paris (FR)

Correspondence Address: Jean-Louis Seugnet;Rhodia Inc.-Legal Department, CN 7500, 8 CEDAR BROOK DRIVE, CRANBURY, NJ, 08512-7500, US

(21) Appl. No.: 11/474,196

(22) Filed: Jun. 23, 2006

(30) Foreign Application Priority Data
Jun. 23, 2005 (FR) 05 06385
Jun. 23, 2005 (FR) 05 06386

(51) Int. Cl. A61K 8/81

(52) U.S. Cl. 424/70.16

(57) ABSTRACT

The present invention relates to a cosmetic composition comprising an ampholytic copolymer and another agent. The amphiphilic polymer comprises particular cationic units and anionic or potentially anionic units. The composition according to the invention satisfies consumer needs for novel compositions.

The present invention relates to a cosmetic composition comprising an ampholytic polymer.

Cosmetic compositions comprising ampholytic copolymers, i.e. copolymers comprising cationic units and anionic or potentially anionic units, are known.

However, there is still a need for cosmetic compositions whose rheology and/or stability and/or feel and/or appearance, especially opacity, sheen, pearlescence, and/or homogeneity, and/or a compromise of these qualities are modified, and/or for cosmetic compositions that modify the appearance of keratin surfaces (especially the skin and/or the hair), and/or their mechanical properties, and/or conditioning, to please at least certain consumers. Consumers are moreover on the lookout for novel products.

The present invention satisfies this need, by proposing a cosmetic composition comprising the following ingredients:
(i) at least 0.05% by weight of an ampholytic copolymer comprising:
   0.1% to 50% by number of cationic units (c) derived from the polymerization of at least one monomeric compound (c) of general formula I: 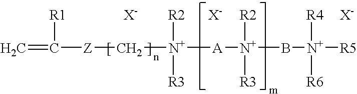
      in which:
      R₁ is a hydrogen atom or a methyl or ethyl group;
      R₂, R₃, R₄, R₅ and R₆, which may be identical or different, are linear or branched C₁-C₆ and preferably C₁-C₄ alkyl, hydroxyalkyl or aminoalkyl groups,
      m is an integer from 0 to 10 and preferably from 0 to 2;
      n is an integer from 1 to 6 and preferably from 2 to 4;
      Z represents a —C(O)O— or —C(O)NH— group or an oxygen atom;
      A represents a group (CH₂)ₚ, p being an integer from 1 to 6 and preferably from 2 to 4;
      B represents a linear or branched C₂-C₁₂ and advantageously C₃-C₆ polymethylene chain, optionally interrupted with one or more heteroatoms or hetero groups, especially O or NH, and optionally substituted with one or more hydroxyl or amino groups, preferably hydroxyl groups;
      X⁻, which may be identical or different, represent counterions;
   anionic or potentially anionic units (a) derived from the polymerization of at least one hydrophilic monomer (a) bearing a function of acidic nature that is copolymerizable with (a), which is anionic or potentially anionic,
   optionally units (n) derived from at least one ethylenically unsaturated monomer (n) of neutral charge, which is copolymerizable with (c) and (a), preferably an ethylenically unsaturated hydrophilic monomer compound of neutral charge bearing one or more hydrophilic groups, which is copolymerizable with (c) and (a),
   the amount of units (a) and optionally (n) being from 50% to 99.9% by number,
(ii) at least 0.1% by weight of an additive chosen from the following compounds:
   ii.1: C₁₆-C₁₈ fatty acid ether or ester agents
   ii.2: synthetic polymers other than the copolymer (a)
   ii.3: polymers of natural origin and derivatives thereof mixtures or combinations thereof
(iii) optionally, at least one agent chosen from
   iii.1: polyorganosiloxanes,
   iii.2: oils other than polyorganosiloxanes, mixtures and combinations thereof
(iv) optionally at least one surfactant,
(v) a cosmetically acceptable vector, preferably water.

Ampholytic copolymer (i)

The ampholytic copolymer (i) comprises:
0.1% to 50% by number of units (c) derived from the polymerization of at least one monomer compound (c) of general formula I: 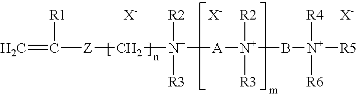
   in which:
   R₁ is a hydrogen atom or a methyl or ethyl group;
   R₂, R₃, R₄, R₅ and R₆, which may be identical or different, are linear or branched C₁-C₆ and preferably C₁-C₄ alkyl, hydroxyalkyl or aminoalkyl groups,
   m is an integer from 0 to 10 and preferably from 0 to 2;
   n is an integer from 1 to 6 and preferably from 2 to 4;
   Z represents a —C(O)O— or —C(O)NH— group or an oxygen atom;
   A represents a group (CH₂)ₚ, p being an integer from 1 to 6 and preferably from 2 to 4;
   B represents a linear or branched C₂-C₁₂ and advantageously C₃-C₆ polymethylene chain, optionally interrupted with one or more heteroatoms or hetero groups, especially O or NH, and optionally substituted with one or more hydroxyl or amino groups, preferably hydroxyl groups;
   X⁻, which may be identical or different, represent counterions;
   units (a) derived from the polymerization of at least one hydrophilic monomer (a) bearing a function of acidic nature that is copolymerizable with (a), which is anionic or potentially anionic,
   optionally units (n) derived from at least one ethylenically unsaturated monomer (n) of neutral charge, which is copolymerizable with (c) and (a), preferably an ethylenically unsaturated hydrophilic monomer compound of neutral charge bearing one or more hydrophilic groups, which is copolymerizable with (c) and (a),
   the amount of units (a) and optionally (n) being from 50% to 99.9% by number.

The ion X⁻ is advantageously chosen from halide, for example chloride, sulfate, methyl sulfate, hydrosulfate, phosphate, citrate, formate and acetate.

The monomer (c) may be prepared, for example, according to the following reaction schemes:

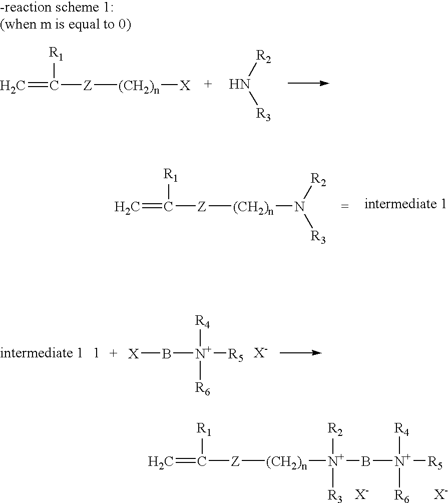

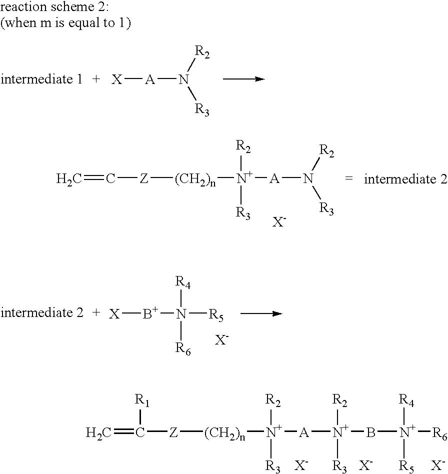

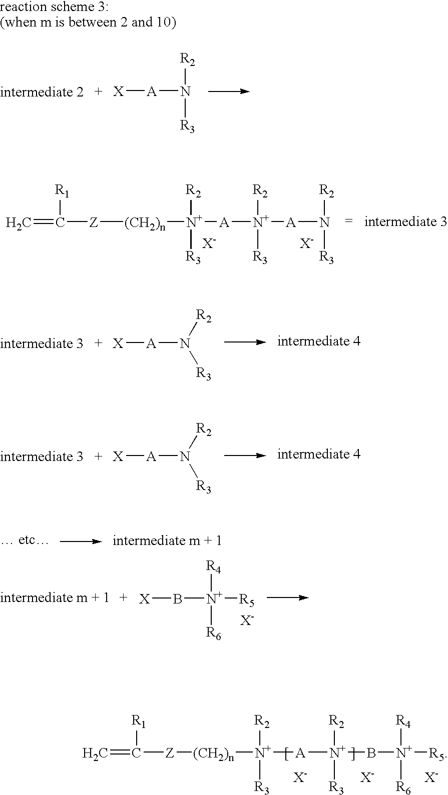

The ampholytic copolymer (i) advantageously has a molecular mass of at least 1000 and advantageously of at least 10 000; it may be up to 20 000 000 and advantageously up to 10 000 000. It is preferably between 500 000 and 5 000 000.

Unless otherwise mentioned, when the term molecular mass is used, it will be the weight-average molecular mass, expressed in g/mol. This may be determined by aqueous gel permeation chromatography (GPC) or by measurement of the intrinsic viscosity in a 1 N solution of NaNO₃ at 30° C.

The copolymer is preferably a random copolymer.

Preferably, in the general formula (I) of the monomer (c),
Z represents C(O)O, C(O)NH or O, and most preferably C(O)NH;
n is equal to 2 or 3 and most particularly 3
m ranges from 0 to 2, it is preferably equal to 0 or 1 and most particularly 0;
B represents: 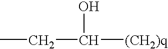
   with q from 1 to 4, preferably equal to 1;
   R₁ to R₆, which may be identical or different, represent a methyl or ethyl group.

The preferred monomer (c) is the DIQUAT having the following formula: 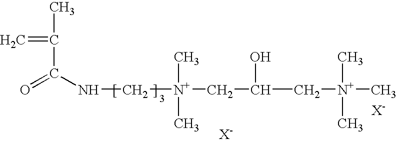
X⁻ representing a chloride or methyl sulfate ion.

Other monomers (c) that are particularly advantageous are:

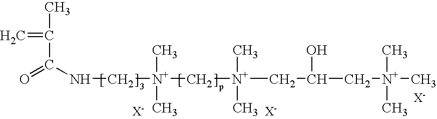

in which p=2 to 4.

The anions X⁻ are especially a halogen anion,. preferably a chloride, sulfonate, sulfate, methyl sulfate, hydrogen sulfate, phosphate, phosphonate, citrate, formate or acetate anion.

The monomers (a) are advantageously monoethylenically unsaturated C₃-C₈ carboxylic, sulfonic, sulfuric, phosphonic or phosphoric acids, anhydrides thereof and water-soluble salts thereof.

Among the preferred monomers (a) that may be mentioned are acrylic acid, methacrylic acid, α-ethacrylic acid, β,β-dimethylacrylic acid, methylenemalonic acid, vinylacetic acid, allylacetic acid, ethylideneacetic acid, propylideneacetic acid, crotonic acid, maleic acid, fumaric acid, itaconic acid, citraconic acid, mesaconic acid, N-methacryloylalanine, N-acryloylhydroxyglycine, sulfopropyl acrylate, sulfoethyl acrylate, sulfoethyl methacrylate, sulfoethyl methacrylate, styrenesulfonic acid, vinylsulfonic acid, vinylphosphonic acid, phosphoethyl acrylate, phosphonoethyl acrylate, phosphopropyl acrylate, phosphonopropyl acrylate, phosphoethyl methacrylate, phosphonoethyl methacrylate, phosphopropyl methacrylate and phosphonopropyl methacrylate, and the alkali metal and ammonium salts thereof.

Among the monomers (n) that may be mentioned are acrylamide, vinyl alcohol, C₁-C₄ alkyl esters of acrylic acid and of methacrylic acid, C₁-C₄ hydroxyalkyl esters of acrylic acid and of methacrylic acid, especially ethylene glycol and propylene glycol acrylate and methacrylate, polyalkoxylated esters of acrylic acid and of methacrylic acid, especially the polyethylene glycol and polypropylene glycol esters, esters of acrylic acid or of methacrylic acid and of polyethylene glycol or polypropylene glycol mono(C₁-C₂₅)alkyl ethers, vinyl acetate, vinylpyrrolidone and methyl vinyl ether.

The ampholytic copolymer (i) comprises from 0.1% to 50% by number of units (c) and from 50% to 99.1% by number of units (a) and optionally (n). Preferably, the copolymer comprises from 10% to 40% of units (c) and from 60% to 90% of units (a) and optionally (n). Moreover, the copolymer advantageously does not comprise any units (n). If the copolymer does comprise units (n), the molar ratio between the units (a) and the units (n) is preferably greater than 1, for example between 1 and 4.

Preferably, the copolymer is such that
the units (c) are derived from a monomer (c) of the following formula: 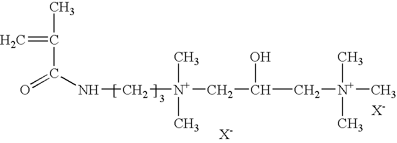
X⁻ representing a chloride or methyl sulfate ion,
the units (a) are derived from acrylic acid,
the polymer does not comprise any units (n),
the numerical ratio between the units (a) and the units (c) is from 50/50 to 90/10.

It is pointed out that the ampholytic copolymers (i) may have a negative, positive or zero mean charge, at the pH of the composition or at the pH of use of the composition. This mean charge is defined by the following equation:

$$Q = \frac{[ c ] {X}_{c} - [ a ] {X}_{a}}{[ c ] {X}_{c} + [ a ] {X}_{a}}$$

in which:
[c] is the molar concentration of units (c),
[a] is the molar concentration of units (a),
X_{c} represents the possible degree of neutralization of the units (c) (in the case where the units (c) are potentially cationic); X_{c}=[BH⁺]/([B]+[BH⁺]),
X_{A} represents the possible degree of neutralization of the units (a) (in the case where the units (a) are potentially anionic); X_{A}=[A⁻]/([AH]+[A⁻]).

The ampholytic copolymers (i) may be obtained according to the known techniques for the preparation of copolymers, especially by free-radical polymerization of ethylenically unsaturated starting monomers that are known compounds or that may be readily obtained by a person skilled in the art using standard synthetic processes of organic chemistry.

Reference may be made especially to the processes described in U.S. Pat. No. 4,387,017 and EP 156 646.

The free-radical polymerization is preferably performed in an oxygen-free environment, for example in the presence of an inert gas (helium, argon, etc.) or nitrogen. The reaction is performed in an inert solvent, preferably methanol or ethanol, and more preferably in water.

The polymerization is initiated by adding a polymerization initiator. The initiators used are the free-radical initiators usually used in the art. Examples include organic peresters (t-butyl peroxypivalate, t-amyl peroxypivalate, t-butyl peroxy-α-ethylhexanoate, etc.); organic compounds of azo type, for example azobisamidinopropane hydrochloride, azobisisobutyronitrile, azobis-2,4-dimethylvaleronitrile, etc.); mineral and organic peroxides, for example hydrogen peroxide, benzyl peroxide and butyl peroxide, etc.; redox initiator systems, for example those comprising oxidizing agents, such as persulfates (especially ammonium or alkali metal persulfates, etc.); chlorates and bromates (including mineral or organic chlorates and/or bromates); reducing agents such as sulfites and bisulfites (including mineral and/or organic sulfites or bisulfites); oxalic acid and ascorbic acid, and also mixtures of two or more of these compounds.

The preferred initiators are water-soluble initiators. Sodium persulfate and azobisamidinopropane hydrochloride are particularly preferred.

As variant, the polymerization may be initiated by irradiation using ultraviolet light. The amount of initiators used is generally an amount that is sufficient to achieve initiation of the polymerization. The initiators are preferably present in an amount ranging from 0.001% to about 10% by weight relative to the total weight of the monomers, and are preferably included in an amount of less than 0.5% by weight relative to the total weight of the monomers, a preferred amount being in the range from 0.005% to 0.5% by weight relative to the total weight of the monomers. The initiator is added to the polymerization mixture either continuously or in a batchwise manner.

When it is desired to obtain copolymers of high molecular mass, it is desirable to add fresh initiator during the polymerization reaction. Gradual or batchwise addition also allows a more efficient polymerization and a shorter reaction time. The polymerization is performed under reaction conditions that are efficient for polymerizing the monomers (c), the monomers (a) and optionally the monomers (n) in an oxygen-free atmosphere. Preferably, the reaction is performed at a temperature ranging from about 30° to about 100° and preferably between 60° and 90° C. The oxygen-free atmosphere is maintained throughout the reaction, for example by maintaining a flow of nitrogen throughout the reaction.

One copolymer that is particularly preferred is the following:

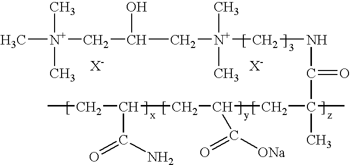

with
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99,9%,
x+y+z=100%, x, y and z representing the molar percentages of units derived, respectively, from acrylamide, acrylic acid (sodium salt) and DIQUAT.

Other polymers are as follows: 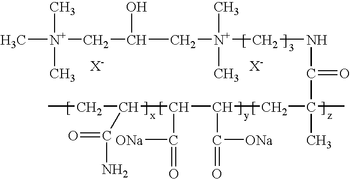
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99.9%. 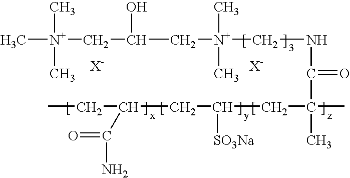
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99.9%. 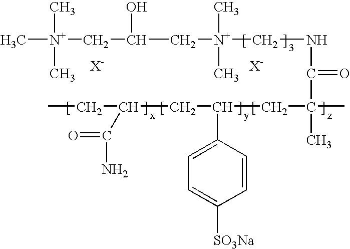
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99.9%. 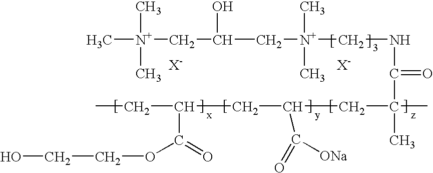
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99.9%. 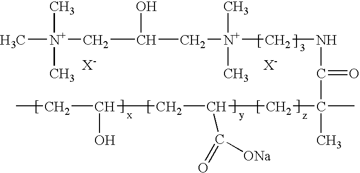
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99.9%. 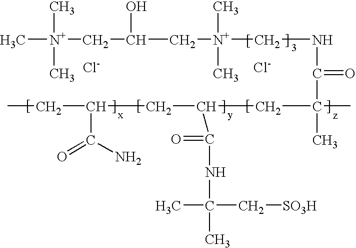
x having a number-average value of from 0 to 50% and preferably from 0 to 30%, preferably equal to 0,
y having a number-average value of from 10% to 95% and preferably from 50% to 70%,
z having a number-average value of from 0.1% to 50% and preferably from 10% to 50%,
the ratio y/z preferably being from about 4/1 to 1/2 and preferably between 4/1 and 1/1,
x+y is from 50% to 99.9%.

The composition comprises at least 0.05% by weight, advantageously from 0.1% to 5%, for example from 0.1% to 0.5% by weight of the polymer. The amounts mentioned above are amounts expressed as solids.

According to one embodiment, the ampholytic copolymer (i) is introduced into the composition separately from the other ingredients, where appropriate in the form of an aqueous solution.

According to one embodiment, the ampholytic copolymer is introduced into the composition in the form of a fluid concentrated ingredient also comprising another ingredient, for example in conditioning agent chosen from the agents (iii). In this case, the composition thus comprises said agent. In particular, it may be a fluid concentrated. ingredient comprising the ampholytic copolymer (i) and a conditioning agent (iii), for which the total weight amount of copolymer (i) and of conditioning agent in the ingredient is at least 10% by weight, preferably at least 20%, preferably at least 50% and preferably at least 60% by weight.

Cosmetically Acceptable Vector

Any cosmetically acceptable vector allowing the ampholytic polymer to be formulated and making it possible to obtain the desired cosmetic composition form, for the intended use, may be used. Various cosmetically acceptable vectors for different types of formulation are known to those skilled in the art.

Examples of cosmetically acceptable vectors that may be mentioned include aqueous vectors (comprising water), alcoholic vectors (comprising an alcohol, for example ethanol, isopropanol, ethylene glycol or polyethylene glycols), propylene glycol, aqueous-alcoholic vectors (comprising a mixture of water and of an alcohol, for example ethanol, isopropanol, ethylene glycol or polyethylene glycols). Certain volatile or non-volatile oils may also be used. Mention may be made, for example, of fluid silicones, such as cyclopentasiloxane, for example Mirasil CM5 sold by Rhodia.

A person skilled in the art knows how to select the vectors that are suitable for the desired types of formulation, and for the intended uses. For example aqueous vectors are generally used for shampoos or shower gels. A propylene glycol vector may be used for compositions in the form of creams. A cyclomethicone vector may be used for makeup compositions, for example for foundations.

In certain cases, the cosmetically acceptable vector may be a mixture with the agent (iii), especially in the case of polyorganosiloxanes. The composition may especially comprise: a polyorganosiloxane acting as vector and as agent (iii). It may comprise two different polyorganosiloxanes, acting as vector and as agent (iii). Naturally, the composition may comprise a polyorganosiloxane and a cosmetically acceptable vector of different nature.

Additive (ii)

The composition comprises at least 0.1% by weight of an additive (ii) chosen from the following compounds:

ii.1: C₁₀-C₃₀ and preferably C16-C₂₂ fatty acid esters of polyols or of monoalcohols, or C₁₀-C₃₀ and preferably C₁₆-C₂₂ fatty alkyl ethers,

ii.2: synthetic polymers other than the copolymer (a),

ii.3: polymers of natural origin and derivatives thereof, mixtures or combinations thereof.

These additives may afford an appearance, texture and/or rheology and/or stability and/or a compromise of some of these properties that are improved compared with compositions comprising the ampholytic copolymer (i) without said additives.

The weight proportion of such additives may typically be from 0.1% to 10% by weight.

Additive ii.1

The additives ii.1 may be chosen from insoluble solid additives forming a network in the composition. These may be fatty acid monoesters and/or diesters of ethylene glycol, the fatty acids preferably being of C₁₆-C₁₈. It may be in particular ethylene glycol distearate (EGDS), for example sold by Rhodia as a concentrate with other ingredients under the name Mirasheen. This compound may typically be present in an amount of from 3% to 10% and preferably from 5% to 8% by weight relative to the composition. It may also be distyryl ether.

The additive ii.1 may be introduced into the composition via any known method, especially by cold mixing, where appropriate in crystalline form, or by hot mixing, where appropriate with subsequent crystallization. The additive ii.1 may be introduced in the form of a mixture with other compounds, especially surfactants.

Among the additives ii.1 that are mentioned are:
distearyl ether
ethylene glycol distearate (EGDS) (INCI: glycol distearate)
polyethoxylated and/or polypropoxylated stearates or distearates, for example PEG-3 distearates, PEG/PPG distearates, PEG-200 distearates, PEG-150 distearates or PEG-100 stearates.

Commercial products that may.be used are especially:
Mirasheen CP 820, Rhodia
Euperlan PK-3000 AM, Cognis
Euperlan PK-771 BENZ, Cognis
Genapol TS, Clariant (INCI: PEG-3 distearate)
Rewopal PEG-6000 DS, Degussa
Carbomer C, Rhodia

Additive ii.2

The additive ii.2 is a synthetic polymer other than the ampholytic copolymer (ii).

It may especially be:

ii.2.a) an optionally crosslinked polyacrylate and/or methacrylate, optionally comprising hydrophobic units, where appropriate in the form of aqueous dispersions in which the polymer becomes dissolved by increasing the pH,

ii.2.b) a cationic or potentially cationic synthetic polymer, or an amphoteric or ampholytic synthetic polymer. Such compounds are especially referenced under the INCI names “Polyquaternium”, listed below.

Among the additives of the type ii.2.a), mention is made especially of:
crosslinked polyacrylates, for example polymers of Carbopol or Carbomer type sold by BF Goodrich or Noveon, Acritamer sold by Rita or Tego Carbomer sold by Goldschmidt. These compounds may be typically present in an amount of from 0.1% to 3% and preferably from 0.3% to 2% by weight relative to the composition. Mention is made in particular of crosslinked copolymers of methacrylic acid and of a C₁-C₄ alkyl acrylate, such as the Carbopol Aqua-SF1 from Noveon;
the acrylate/aminoacrylate/PEG-20 C₁₀-C₃₀ alkyl itaconate copolymers sold by National Starch under the name Structure Plus. These compounds may typically be present in an amount of from 0.1% to 3% and preferably from 0.3% to 2% by weight relative to the composition;
viscosifiers, gelling agents or texturing agents, for instance anionic acrylic copolymers of Aculyn type sold by ISP or Rohm & Haas.

As commercial compounds that may be used as additive ii.2.a, mention is made of:
Carbopol ETD-2020, Noveon
Carbopol Aqua SF-1, Noveon
Carbopol 980, Noveon
Aculyn 22, Rohm & Haas
Structure Plus, National Starch.

Additives ii.3

The additives ii.3 are natural polymers or derivatives of these natural polymers. They may especially be cationic, nonionic or anionic derivatives, which are, where appropriate, hydrophobic. They may be, for example, polysaccharides or polysaccharide derivatives, keratin derivatives, or proteins or protein derivatives. Cationic derivatives of natural polymers are especially referenced under the INCI name “Polyquaternium”, listed below.

Mention may be made especially of polysaccharides and noncationic derivatives thereof, such as cellulose derivatives, for instance hydroxypropylcellulose, carboxymethylcellulose, nonionic guar derivatives, for instance hydroxypropyl guar (for example the Jaguar HP products sold by Rhodia), locust bean gum, tara gum or cassia gum, xanthan gum (for example the Rhodicare products sold by Rhodia), succinoglycans (for example Rheozan sold by Rhodia), alginates, carrageenans, chitin derivatives or any other polysaccharide with a texturing function. These polysaccharides and derivatives thereof may be incorporated alone or in synergistic combination with other polysaccharides. These compounds may typically be present in an amount of from 0.1% to 3% and preferably from 0.3% to 1% by weight relative to the composition.

As additives of the type iii.3, mention may be made in particular of:
xanthan gum,
succinoglycan,
hydroxyethylcellulose,
hydroxypropyl guar,
hydrolyzed keratins.

Additives ii.2.b) or ii.3 are especially the polymers of the Polyquaternium type according to the INCI terminology that is familiar to those skilled in the art, chosen, for example, from the polymers of table I below.

TABLE 1

| | | Chemical nature and/or | Commercial |
|---|---|---|---|
| INCI name | Type | CAS number | compounds |
| Polyquaternium-2 | b2 | CAS 63451-27-4 | Mirapol A15, Rhodia |
| Polyquaternium-4 | a1 | CAS 92183-41-0 | Celquat L200, H100, |
| | | | National Starch |
| Polyquaternium-5 | b2 | CAS 26006-22-4 | |
| Polyquaternium-6 | b2 | DADMAC polymer | Merquat 1000, Nalco, |
| | | CAS 26062-79-3 | Mirapol 100, Rhodia |
| Polyquaternium-7 | b2 | Copolymer of DADMAC | Merquat 5500, Nalco; |
| | | and of acrylamide | Mirapol 550, Rhodia |
| | | CAS 26590-05-6 | |
| Polyquaternium-10 | a1 | Hydroxyethylcellulose | Polymer JR 400, |
| | | modified with | Amerchol; Celquat |
| | | trimethylammoniums | SC230M or SC-240C, |
| | | | National Starch |
| Polyquaternium-11 | b2 | Copolymers of vinylpyrrolidone | Gafquat 755N, ISP; |
| | | and of quaternized | Luviquat PQ11 PN, |
| | | dimethylaminoethyl | BASF |
| | | methacrylate | |
| Polyquaternium-16 | b2 | CAS 29297-55-0 | Luviquat HM 552, |
| | | | Luviquat FC 370, |
| | | | BASF |
| Polyquaternium-17 | b2 | CAS 90624-75-2 | Mirapol AD1, Rhodia |
| Polyquaternium-19 | b2 | CAS 110736-85-1 | |
| Polyquaternium-22 | b2 | Copolymer of DADMAC | Merquat 280, 281, |
| | | and of acrylic acid | 298, Nalco |
| Polyquaternium-24 | a1 | Hydroxyethylcellulose | Quatrisoft LM200, |
| | | modified with quaternary | Amerchol |
| | | ammoniums containing | |
| | | long alkyl chains | |
| Polyquaternium-27 | b2 | | Merquat 2001, Nalco |
| Polyquaternium-28 | b2 | Copolymer of vinylpyrrolidone | Gafquat HS 100, |
| | | and of MAPTAC | BASF |
| Polyquaternium-29 | b2 | Chitosan derivative | Kytamer KCO, |
| | | modified with propylene | Amerchol, |
| | | oxide and quaternized with | Lexquat CH |
| | | epichlorohydrin | |
| Polyquaternium-31 | b2 | CAS 136505-02-7 and | Hypan HQ |
| | | 139767-67-7 | |
| Polyquaternium-32 | b2 | CAS 254429-19-7 | |
| Polyquaternium-37 | b2 | CAS 35429-19-7 | |
| Polyquaternium-39 | b2 | | Merquat 3300, 3331, |
| | | | Nalco |
| Polyquaternium-44 | b2 | | Luviquat Care, BASF |
| Polyquaternium-46 | b2 | copolymers of vinylcaprolactam, | Luviquat Hold, BASF |
| | | vinylpyrrolidone, and | |
| | | cationized vinylimidazole | |
| Guar hydroxypropylammonium | a1 | | Jaguar C135, C14S, |
| chloride | | | C17, Excel, Rhodia |
| Hydroxypropyl gural | a1 | | Jaguar C162, Rhodia |
| hydroxypropylammonium | | | |
| chloride | | | |
| Polyquaternium-67 | a1 | Hydroxyethylcellulose | Softcat SL, Amerchol |
| | | modified with quaternary | |
| | | ammoniums containing | |
| | | long alkyl chains and with | |
| | | short-chain quaternary | |
| | | ammoniums | |
| Polymethacrylamidopropyltrimonium | | MAPTAC polymer | Polycare 133, Rhodia |
| chloride | | | |
| Acrylamidopropyltrimonium | | | Salcare SC-60, Ciba |
| chloride/acrylamide | | | |
| copolymer | | | |

Agent (iii)

The composition may comprise at least one agent (iii) chosen from
iii.1: polyorganosiloxanes,
iii.2: oils other than polyorganosiloxanes, especially plant, mineral or animal oils, or derivatives thereof,
mixtures or combinations thereof.

This agent may especially give a conditioning effect to keratin materials, especially the skin and/or the hair.

Agent iii.2

The agent iii.1 is a polyorganosiloxane. Polyorganosiloxanes are also referred to as silicones. The terms “silicone” and “polyorganosiloxane” mean any organosiloxane compound comprising alkyl groups (for example methyl) and/or functionalized with groups other than alkyl groups.

The polyorganosiloxane is advantageously (in shampoos and hair conditioners in particular) a nonvolatile water-insoluble polyorganosiloxane. It advantageously has a viscosity of between 1000 and 2 000 000 mPa·s and preferably between 5000 and 1 000 000 mPa·s (at 25° C.). The polyorganosiloxane may especially be a polydimethylorganosiloxanesiloxane (“PDMS”, INCI name: dimethicone) or a polyorganosiloxane containing amine groups (for example Amodimethicone according to the INCI name), quaternary ammonium groups (for example the silicones Quaternium 1 to 10 according to the INCI name), hydroxyl groups (terminal or nonterminal), polyoxyalkylene groups, for example polyethylene oxide and/or polypropylene oxide (as end groups, as a block in a PDMS chain, or as grafts) or aromatic groups, or several of these groups.

The polyorganosiloxanes that are useful in the cosmetics field and the characteristics thereof are known to those skilled in the art.

The polyorganosiloxanes (silicones) are preferably present in the composition or in the concentrated ingredient in emulsion form (liquid droplets of silicone dispersed in the aqueous phase). The emulsion may especially be an emulsion with a mean droplet size of greater than or equal to 2 μm, or with a mean droplet size of between 0.15 μm and 2 μm, or with a mean droplet size of less than or equal to 0.15 μm.

The droplets of the emulsion may be of more or less large size. Reference may thus be made to microemulsions, miniemulsions or macroemulsions. In the present patent application, the term “emulsion” especially covers all these types of emulsion. Without wishing to be bound to any theory, it is pointed out that microemulsions are generally thermodynamically stable systems, generally comprising large amounts of emulsifiers such as surfactants c). The other emulsions are generally systems in thermodynamically unstable state, conserving for a certain time, in metastable state, the mechanical energy supplied during the emulsification. These systems generally comprise smaller amounts of emulsifiers.

The emulsions may be obtained by mixing an outer phase, which is preferably aqueous, polyorganosiloxane, polymer for aiding deposition and, in general, an emulsifier, followed by emulsification. This process may be referred to as in-situ emulsification.

The microemulsion droplet size may be measured on an emulsion prepared prior to its introduction into the cosmetic composition, by dynamic light scattering (DQEL), for example as described below. The apparatus used consists, for example, of a Spectra-Physics 2020 laser, a Brookhaven 2030 correlator and the associated computerware. Since the sample is concentrated, it is diluted in deionized water and filtered through a 0.22 μm filter in order finally to be at 2% by weight. The diameter obtained is an apparent diameter. The measurements are taken at angles of 90° and 135°. For the size measurements, besides the standard cumulative analysis, three exploitations of the self-correlation function are used (exponential sampling or EXPSAM described by Prof. Pike, the “Non-Negatively Constrained Least Squares” or NNLS method, and the CONTIN method described by Prof. Provencher), which each give a size distribution weighted by the scattered intensity, rather than by the mass or the number. The refractive index and the viscosity of water are taken into account.

According to one useful embodiment, the composition and/or the concentrated ingredient is (are) transparent. The composition and/or the concentrated ingredient may, for example, have a transmittance of at least 90% and preferably of at least 95%, at a wavelength of 600 nm, for example measured using a Lambda 40 UV-Vis spectrometer, at a concentration of 0.5% by weight in water.

According to another particular embodiment, the composition or the concentrated ingredient is an emulsion whose mean droplet size is greater than or equal to 0.15 μm, for example greater than 0.5 μm, or 1 μm, or 2 μm, or 10 μm, or 20 μm, and preferably less than 100 μm. The droplet size may be measured on an emulsion prepared prior to its introduction into the cosmetic composition, by optical microscopy and/or laser granulometry (Horiba LA-910 laser scattering analyzer). In this embodiment, the composition and/or the concentrated ingredient preferably comprise(s) a proportion of less than 10% by weight of emulsifier relative to the weight of polyorganosiloxane.

Among the water-soluble silicones of the composition that may be mentioned, inter alia, are dimethicone copolyols (Mirasil DMCO sold by the company Rhodia Chimie).

As regards silicones in the form of water-insoluble dispersions or emulsions, nonvolatile water-insoluble organopolysiloxanes may appropriately be used, among which mention may be made of polyalkylsiloxane, polyarylsiloxane, and polyalkylarylsiloxane oils, gums or resins or nonvolatile water-insoluble functionalized derivatives thereof, or mixtures thereof.

Said organopolysiloxanes are considered as being water-insoluble and nonvolatile when their solubility in water is less than 50 g/liter and their intrinsic viscosity is at least 3000 mPa·s, at 25° C.

Examples of nonvolatile water-insoluble organopolysiloxanes or silicones that may be mentioned include silicone gums, for instance the diphenyl dimethicone gum sold by the company Rhodia Chimie, and preferably polydimethylorganosiloxanes with a viscosity at least equal to 6×10⁵ mPa·s, at 25° C., and even more preferentially those with a viscosity of greater than 2×10⁶ mPa·s, at 25° C., such as Mirasil DM 500000® sold by the company Rhodia Chimie.

According to the invention, the nonvolatile water-insoluble organopolysiloxane or silicone is in a form dispersed in the cosmetic composition or the concentrated ingredient containing it.

Among these low-viscosity silicones, mention may be made of cyclic volatile silicones and polydimethylorganosiloxanes of low mass.

It is also possible to use functionalized silicone derivatives, for instance amine derivatives directly in the form of emulsions or starting with a preformed microemulsion. These may be compounds known as amino silicones or hydroxy silicones. Mention is made, for example, of the oil Rhodorsil amine 21637 (Amodimethicone) sold by the company Rhodia, and dimethiconol.

As polyorganosiloxanes that may be used mention is made especially of:
polyorganosiloxanes comprising units —Si(CH₂)₂O— and units —SiY(CH₂)O— in which Y is a —(CH₂)₃—NH(CH₂)₂—NH₂ or —(CH₂)₃—NH₂ group,
polyorganosiloxanes comprising units —Si(CH₂)₂O — and end units —HO—Si(CH₂)₂— and/or non-end units —Si(CH₂)(OH)O—
polyorganosiloxanes comprising units —Si(CH₂)₂O— and units —SiY(CH₂)O— in which Y is
   -L^{X}-Z^{x}-Palk in which L^{X} is a divalent bonding group, preferably an alkyl group, Z^{X} is a covalent bond or a divalent connecting group comprising a heteroatom, Palk is a group of formula [OE]ₛ-[OP]ₜ—X′, in which OE is a group of formula —CH₂—CH₂—O—, OP is a group of formula —CH₂—CHCH₃—O— or —CHCH₃—CH₂—O—, X′ is a hydrogen atom or a hydrocarbon-based group, s is a mean number greater than 1, and t is a mean number greater than or equal to 0,
polyorganosiloxanes whose chain comprises at least one block comprising units of formula —Si(CH₂)₂O— and at least one block —[OE]₅-[OP]ₜ—,
polyorganosiloxanes comprising units —Si(CH₂)₂O— and/or units —Si(CH₂)RO— and/or —SiR₂O— and/or R—Si(CH₂)₂O— and/or H₃C—SiR₂O— and/or R—SiR₂O— in which R, which may be identical or different, is an alkyl group other than a methyl group, an aryl group, an alkyl group, an alkylaryl group or an aralkyl group.

The following commercially available ingredients may especially be used as polyorganosiloxanes:
Mirasil DM 500000, Rhodia (INCI: Dimethicone), for example in the form of an emulsion with a particle size of 0.6 μm or 0.9 μm,
Mirasil DME-2, Rhodia (INCI: dimethicone)
Mirasil DME30, Rhodia (INCI: dimethicone)
Mirasil ADM-E, Rhodia (INCI: amodimethicone)
Dow Corning 1784 HVF, Dow Corning (INCI: Dimethiconol)
Dow Corning 1784 HMW, Dow Corning (INCI: Divinyldimethicone/dimethicone)
Mirasil DMCP-93, Rhodia (INCI: PEG/PPG-10/2 dimethicone)
Parsol SLX, DSM (INCI: Polysilicone-15)
Mirasil SM, Rhodia (INCI: Simethicone)
Mirasil DMCO, Rhodia (INCI PEG/PPG-22/24 Dimethicone)
Mirasil DM 100000, Rhodia (INCI: Dimethicone)
DC200 fluid 60000, Dow Corning (INCI: Dimethicone)
DC200 fluid 300000, Dow Corning (INCI: Dimethicone).

Agent iii.2

Among the plant oils and derivatives thereof that may especially be mentioned are: almond oil (sweet almond oil), anhydrous lanolin oil, apricot kernel oil, avocado oil, castor oil, jojoba oil, olive oil, groundnut oil, sesame seed oil, sunflower oil, corn oil, cottonseed oil, hydrogenated vegetable oils, soybean oil, sulfonated castor oil, coconut oil, cocoa butter, wheatgerm oil, Aloe vera, grapeseed oil, hazelnut oil, macadamia nut oil, “St-Jean protuberance” oil, walnut oil, hazelnut oil, borage oil, peach kernel oil, virgin coconut oil, baobab oil, avocado butter, palm oil, palm kernel oil, flax oil, copra oil, babassu oil and wheatgerm oil.

Among the oils of animal origin that may be mentioned, inter alia, are sperm whale oil, whale oil, seal oil, sardine oil, herring oil, shark oil, cod liver oil; pig fat or sheep fat (tallow).

As regards mineral oils, mention may be made, inter alia, of naphthenic oils and paraffinic oils (petroleum jelly or petrolatum). Mention may also be made of perhydrosqualene and squalene.

Surfactants

The composition may comprise at least one surfactant (iv). It may be a mixture of different surfactants. The surfactants may be anionic, cationic, nonionic or amphoteric surfactants, or mixtures or combinations. The surfactants included in the composition preferably comprise at least one anionic or cationic surfactant. The surfactants may also comprise amphoteric surfactants (true amphoteric or zwitterionic surfactants) or neutral surfactants (nonionic surfactants). The compositions comprising at least one anionic surfactant and at least one amphoteric surfactant are particularly advantageous, especially for reasons of softness. The total amount of surfactants in the composition is between 0 and 30% by weight.

For rinse-off or leave-on the hair conditioning compositions, the surfactant is preferably absent or present in an amount of less than 5% by weight, and it may preferably be a cationic surfactant.

For compositions intended for treating the hair, for instance shampoos, the surfactant content is advantageously between 10% and 20% by weight. Such compositions may comprise salts, for example sodium or ammonium chloride, advantageously in a content of less than 3% by weight.

For compositions intended for treating the skin, for instance shower gels, the surfactant content is advantageously between 5% and 15% by weight. Such compositions also preferably comprise at least 2% by weight of salts, for example sodium or ammonium chloride.

For hair conditioners, the surfactant content may be less than 5% by weight.

The weight proportion of anionic surfactants relative to the total amount of surfactants is preferably greater than 50% and preferentially greater than 70%.

The anionic surfactants may be chosen from the following surfactants:
alkyl ester sulfonates, for example of formula R—CH(SO₃M)-CH₂COOR′, or alkyl ester sulfates, for example of formula R—CH(OSO₃M)-CH₂COOR′, in which R represents a C₈-C₂₀ and preferably C₁₀-C₁₆ alkyl radical, R′ a C₁-C₆ and preferably C₁-C₃ alkyl radical and M an alkaline-earth metal cation, for example sodium, or an ammonium cation. Mention may be made most particularly of methyl ester sulfonates whose radical R is of C₁₄-C₁₆;
alkylbenzenesulfonates, more particularly of C₉-C₂₀, primary or secondary alkylsulfonates, especially of C₈-C₂₂, and alkylglyceryl sulfonates;
alkyl sulfates, for example of formula ROSO₃M, in which R represents a C₁₀-C₂₄ and preferably C₁₂-C₂₀ alkyl or hydroxyalkyl radical; M represents a cation of the same definition as above;
alkyl ether sulfates, for example of formula RO(OA)ₙSO₃M in which R represents a C₁₀-C₂₄ and preferably C₁₂-C₂₀ alkyl or hydroxyalkyl radical; OA representing an ethoxylated and/or propoxylated group; M representing a cation of the same definition as above, n generally ranging from 1 to 4, for instance lauryl ether sulfate with n=2;
alkylamide sulfates, for example of formula RCONHR′OSO₃M in which R represents a C₂-C₂₂ and preferably C₆-C₂₀ alkyl radical, R′ represents a C₂-C₃ alkyl radical, M representing a cation of the same definition as above, and also the polyalkoxylated (ethoxylated and/or propoxylated) derivatives thereof (alkylamido ether sulfates)
saturated or unsaturated fatty acid salts, for example those of C₈-C₂₄ and preferably of C₁₄-C₂₀ and of an alkaline-earth metal cation, N-acyl N-alkyltaurates, alkylisethionates, alkylsuccinamates and alkylsulfosuccinates, sulfosuccinate monoesters or diesters, N-acyl sarcosinates and polyethoxycarboxylates;
phosphate monoesters and diesters, for example having the following formula: (RO)ₓ—P(═O)(OM)ₓ in which R represents an alkyl, alkylaryl, arylalkyl or aryl radical, which are optionally polyalkoxylated, x and x′ being equal to 1 or 2, on condition that the sum of x and x′ is equal to 3, M representing an alkaline-earth metal cation.

The nonionic surfactants may be chosen from the following surfactants:
alkoxylated fatty alcohols
alkoxylated triglycerides
alkoxylated fatty acids
alkoxylated sorbitan esters
alkoxylated fatty amines
alkoxylated bis(1-phenylethyl)phenols
alkoxylated tris(1-phenylethyl)phenols
alkoxylated alkylphenols
products resulting from the condensation of ethylene oxide with a hydrophobic compound resulting from the condensation of propylene oxide with propylene glycol, such as the Pluronic products sold by BASF;
products resulting from the condensation of ethylene oxide with the. compound resulting from the condensation of propylene oxide with ethylenediamine, such as the Tetronic products sold by BASF;
alkylpolyglycosides, for instance those described in U.S. Pat. No. 4,565,647, or alkylglucosides;
fatty acid amides, for example of C₈-C₂₀, especially fatty acid monoalkanolamides, for example cocamide MEA or cocamide MIPA;

The amphoteric surfactants (true amphoteric surfactants comprising an ionic group and a potentially ionic group of opposite charge, or zwitterionic surfactants simultaneously comprising two opposite charges) may be chosen from the following surfactants:
betaines in general, especially carboxy betaines, for example lauryl betaine (Mirataine BB from the company Rhodia) or octyl betaine or cocobetaine (Mirataine BB-FLA from Rhodia); and amidoalkyl betaines, for instance cocamidopropyl betaine (CAPB) (Mirataine BDJ from the company Rhodia or Mirataine BET C-30 from Rhodia);
sulfobetaines or sultaines, for instance cocamidopropyl hydroxy sultaine (Mirataine CBS from the company Rhodia);
alkylamphoacetates and alkylamphodiacetates, for instance those comprising a coco or lauryl chain (Miranol C2M, C32 and L32 especially, from the company Rhodia);
alkylamphopropionates or alkylamphodipropionates, (Miranol C2M SF);
alkyl amphohydroxypropyl sultaines (Miranol CS)
alkylamine oxides, for example lauramine oxide (INCI).

The cationic surfactants may be chosen from primary, secondary or tertiary, optionally polyethoxylated fatty amine salts, quaternary ammonium salts such as tetraalkylammonium, alkylamidoalkylammonium, trialkylbenzylammonium, trialkyl-hydroxyalkylammonium or alkylpyridinium chlorides or bromides, imidazoline derivatives and amine oxides of cationic nature. An example of a cationic surfactant is cetrimonium chloride or bromide (INCI).

Examples of useful compositions that may be mentioned include:
the “sodium” compositions for shampoos typically comprising 12% to 16% by weight of sodium alkyl ether sulfate (for example sodium lauryl ether sulfate “SLES”) or a mixture of sodium alkyl ether sulfate and of sodium alkyl sulfate (for example sodium lauryl sulfate “SLS”), 1% to 3% of an amphoteric surfactant (for example cocoamidopropyl betaine “CAPB”), 0.5% to 2% of a salt (for example sodium chloride);
the “ammonium” compositions for shampoos typically comprising 12% to 16% by weight of ammonium alkyl ether sulfate (for example ammonium lauryl ether sulfate “ALES”) or of a mixture of ammonium alkyl ether sulfate and of ammonium alkyl sulfate (for example ammonium lauryl sulfate “ALS”), 1% to 3% of an amphoteric surfactant (for example cocoamidopropyl betaine “CAPB”), 0 to 2% of a salt (for example ammonium chloride);
the “sodium” compositions for shower gels typically comprising 6% to 10% by weight of sodium alkyl ether sulfate (for example sodium lauryl ether sulfate “SLES”) or of a mixture of sodium alkyl ether sulfate and of sodium alkyl sulfate (for example sodium lauryl sulfate “SLS”), 1% to 3% of an amphoteric surfactant (for example cocoamidopropyl betaine “CAPB”), 2% to 4% of a salt (for example sodium chloride);
the “sodium” compositions for shower gels typically comprising 6% to 10% by weight of ammonium alkyl ether sulfate (for example ammonium lauryl ether sulfate “ALES”) or a mixture of ammonium alkyl ether sulfate and of ammonium alkyl sulfate (for example ammonium lauryl sulfate “ALS”), 1% to 3% of an amphoteric surfactant (for example cocoamidopropyl betaine “CAPB”), 0 to 4% of a salt (for example ammonium chloride).

Other Agents

The composition may especially comprise salts, for example sodium chloride or ammonium chloride. The amount of salts may vary as a function of the desired viscosity, and may be, for example, between 0.05% and 5%.

The composition according to the invention may comprise humectants, for example glycerol, sorbitol, urea, collagen, gelatin, Aloe vera or hyaluronic acid, or water-soluble volatile solvents, for instance ethanol or propylene glycol, the contents of which may be up to 60% by weight of the composition. Glycerol may be used for instance, for example in an amount of from 0.1% to 2%.

The composition according to the invention may comprise fragrances, in an amount appreciated by the formulator, for example at least 0.1%.

The composition according to the invention may comprise preserving agents, in an amount appreciated by the formulator.

The composition according to the invention may comprise dyes, in an amount appreciated by the formulator.

The composition according to the invention may especially comprise other additives occasionally known as active materials. Mention is made, for example, of antioxidant compounds, compounds that eliminate free radicals, compounds for protecting against light radiation, and antidandruff compounds. Bactericides or fungicides may be incorporated into the cosmetic composition, for example, in order to improve the disinfection of the skin, for instance triclosan; antidandruff agents, especially such as zinc pyrithione or octopirox; insecticides, for instance natural or synthetic pyrethroids. The cosmetic compositions may especially contain agents for protecting the skin and/or the hair against attack by sunlight and UV rays. Thus, the compositions may comprise sunscreens, which are chemical compounds that strongly absorb UV radiation, for instance the compounds permitted in European directive No. 76/768/EEC, its appendices and subsequent amendments of this directive. Selenium sulfide and salicylic acid may also be used as active material.

When the various constituents of the cosmetic composition exhibit excessively poor solubility in the composition or when they are in solid form at room temperature, said constituents may also be dissolved in an organic vehicle, for instance mineral or natural oils, silicone derivatives or waxes, or alternatively may be encapsulated in matrices, for instance polymers of latex type.

Metal sequestrants, more particularly those that sequester calcium, for instance citrate ions, may also advantageously be added to these compositions.

Humectants may also be incorporated into the cosmetic compositions forming the subject of the invention, among which are, inter alia,

To further reduce the irritation or attack of the scalp, water-soluble or water-dispersible polymers may also be added, for instance collagen or certain non-allergenic derivatives of animal or plant proteins (for example wheat protein hydrolyzates), natural hydrocolloids (guar gum, locust bean gum, tara gum, etc.) or hydrocolloids derived from fermentation processes, and derivatives of these polycarbohydrates, for instance modified nonionic celluloses, for instance hydroxyethylcellulose, or modified anionic celluloses, for instance carboxymethylcellulose; guar derivatives or locust bean gum derivatives, for instance the nonionic derivatives thereof (for example hydroxypropyl guar) or the anionic derivatives thereof (carboxymethyl guar and carboxymethylhydroxypropyl guar).

Mineral powders or particles, for instance calcium carbonate, sodium bicarbonate, calcium dihydrogen phosphate, mineral oxides in powder form or in colloidal form (particles less than about 1 micrometer in size, occasionally a few tens of nanometers), for instance titanium dioxide, silica, aluminum salts generally used as antiperspirants, kaolin, talc, clays and derivatives thereof; etc., may be added in combination to these compounds.

Preserving agents, for instance methyl, ethyl, propyl and butyl esters of p-hydroxybenzoic acid, sodium benzoate, Germaben® or any chemical agent for preventing the proliferation of bacteria or molds that is conventionally used in cosmetic compositions may also be introduced into the aqueous cosmetic compositions according to the invention, generally to a proportion of from 0.01% to 3% by weight.

The amount of these products is usually adjusted to prevent any proliferation of bacteria, molds or yeasts in the cosmetic compositions.

As an alternative to these chemical agents, it may occasionally be possible to use agents that modify the water activity and that greatly increase the osmotic pressure, for instance carbohydrates or salts.

To protect the skin and/or the hair against attack from sunlight and UV rays, organic or mineral sunscreens may be added to the compositions, for example mineral particles, for instance zinc oxide, titanium dioxide or cerium oxides, in powder form or in the form of colloidal particles, alone or as a mixture. These powders may optionally be surface-treated to increase the efficacy of their anti-UV action or to facilitate their incorporation into the cosmetic formulations, or to prevent surface photoreactivity. The organic sunscreens may especially be introduced into the polyorganosiloxane, if it is present in the composition.

One or more fragrances, colorants chosen from, among which mention may be made of the products described in appendix IV (“List of coloring agents allowed for use in cosmetic products”) of European directive No. 76/768/EEC of 27 Jul. 1976, known as the Cosmetic Directive, and/or opacifiers, for instance pigments, may be added to these ingredients, if necessary, with the aim of increasing the comfort during the use of the composition by the consumer.

Although this is not obligatory, the composition may also contain viscosifying or gelling polymers so as to adjust the texture of the composition, for instance the crosslinked polyacrylates (Carbopol sold by Goodrich) already mentioned above, noncationic cellulose derivatives, for instance hydroxypropylcellulose or carboxymethylcellulose, guars and nonionic derivatives thereof, xanthan gum and its derivatives, used alone or in combination, or the same compounds, generally in the form of water-soluble polymers modified with hydrophobic groups covalently bonded to the polymer skeleton, as described in patent WO 92/16187 and/or water to bring the total of the constituents of the formulation to 100%.

The cosmetic compositions forming the subject of the invention may also contain polymeric dispersants in an amount of about 0.1-7% by weight, to control the calcium and magnesium hardness, these being agents such as:
water-soluble polycarboxylic acid salts with a weight-average molecular mass of about from 2000 to 100 000g/mol, obtained by polymerization or copolymerization of ethylenically unsaturated carboxylic acids such as acrylic acid, maleic acid or anhydride, fumaric acid, itaconic acid, aconitic acid, mesaconic acid, citraconic acid or methylenemalonic acid, and most particularly polyacrylates with a weight-average molecular mass of about from 2000 to 10 000 g/mol (U.S. Pat. No. 3,308,067), copolymers of acrylic acid and of maleic anhydride with a weight-average molecular mass of about from 5000 to 75 000 g/mol (EP-A-66 915);
polyethylene glycols with a weight-average molecular mass of about from 1000 to 50 000 g/mol.

The composition may comprise any other compound that may especially be used in cosmetic compositions intended to be rinsed out (shampoo, shower gel, conditioner, etc.) or not intended to be rinsed out. It is not excluded for certain compounds to exert several functions. Such compounds may appear in several sections of the present patent application.

Examples that may be mentioned include sequestrants, softeners, foam modifiers, colorants, nacreous agents (pearlizers), moisturizers, antidandruff or antiseborrheic agents, suspension agents, emulsifiers, ceramides, pseudoceramides, electrolytes, fatty acids, fatty acid esters, hydroxy acids, thickeners, fragrances, preserving agents, organic or mineral sunscreens, proteins and derivatives thereof, vitamins, stabilizers and rheology modifiers. Some of these compounds are detailed below.

Composition

According to one embodiment, the pH of the composition may be between 3.5 and less than 5.5. According to another embodiment, it may be between 5.5 and 12, preferably between 5.5 and 9 and preferably between 5.5 and 7.5. The pH obviously depends on the compounds present in the composition. pH regulators, acids or bases, may obviously be used in the composition, for example citric acid, or sodium hydroxide, potassium hydroxide or ammonium hydroxide.

The cosmetic composition may especially comprise:
from 0.1% to 0.5% by weight of the copolymer (i)
from 0.15% to 7% by weight of the additives (ii).

Advantageously, it may comprise from 0.1% to 60%, often from 0.1% to 15%, or from 0.1% to 5%, by weight of a polyorganosiloxane (iii).

Advantageously, it may comprise from 0.1% to 60% and often from 0.1% to 15% of an oil other than polyorganosiloxanes.

The composition according to the invention may be formulated in various forms, which depend on the appearance that it is desired to give it, the sensory properties (viscosity, feel, permanence, etc.) that it is desired to give it, and, obviously, the intended use thereof. The various types of formulation and the various uses are modulated by the nature and the amount of the ingredients present in the composition, and are known to those skilled in the art.

Thus, the composition may be formulated in the form of gels, more or less viscous fluids, milks, creams, oils, sprays, mousses, gels in wand form (sticks), pastes, lotions, dye concentrates, etc.

The compositions may be chosen especially from the compositions listed in the table below, with types of formulation and uses also listed in table (I) below. For these compositions, physicochemical forms, types of formulation and uses, reference may be made to more detailed sections of the present patent application.

TABLE I

| Composition | Type of formulation | Use |
|---|---|---|
| Shampoos | Fluid | Hair cleansing and/or haircare |
| | | and/or temporary dyeing |
| | | and/or coloration fixing, with |
| | | rinsing |
| Hair | Fluid | Haircare and/or disentangling |
| conditioner | | and/or styling and/or |
| | | temporary dyeing and/or |
| | | coloration fixing and/or |
| | | conditioning and/or conditioning |
| | | after dyeing, with or |
| | | without rinsing |
| Gel-douche | Fluid or gel | Skin cleansing and/or skincare |
| Hair mask | Very viscous fluid | Haircare |
| Antisun | Cream | UV protection of the skin |
| cream | | |
| Antisun milk | Milk | UV protection of the skin |
| Antisun oil | Oil | UV protection of the skin |
| Antisun spray | Fluid | UV protection of the skin |
| Care cream | Cream | Skincare |
| Makeup | Cream or fluid or gel | Care and/or cleansing of the |
| remover | | skin and/or the eyelashes |
| Makeup | Cream, fluid, | Coloring of the skin or the |
| | mascara, powder, | eyelashes |
| | gel, stick | |
| Deodorant | Aerosol, gel, stick, | Reduction of perspiration |
| | substance that can | effects, applied to the skin |
| | be applied using a | |
| | roll-on applicator | |
| Shaving foam | Very fluid liquid or gel | Preparation for shaving |
| | forming a foam after | |
| | aerosol spraying | |
| Styling or | Fluid | Hair shaping |
| fixing spray | | |
| Styling or | Gel | Hair shaping |
| fixing gel | | |
| Styling or | Very fluid liquid or gel | Hair shaping |
| fixing foam | forming a foam after | |
| | aerosol spraying | |
| Dye | Gel or viscous liquid | Permanent or semipermanent |
| composition | | dyeing |

Among the uses of the compositions, mention may be made of uses in which the composition is intended to be rinsed off and uses in which the composition is not intended to be rinsed off.

In the cosmetic composition, the agent (iii) is advantageously present in the form of an emulsion preferably with a droplet size. The physical form may depend on the other ingredients of the composition and on the process for its preparation. High shears may lead, for example, to small droplet sizes.

The compositions are preferably compositions intended to be rinsed off. Such a composition may be, for example, a shampoo, a shower gel or a hair conditioner. However, it may be a haircare composition that is not intended to be rinsed off, for example a hair conditioner not intended to be rinsed off, a disentangling milk, a disentangling lotion, a smoothing lotion, a cuticle coat, a styling and/or restyling haircare product, an antisun product, a care cream, a makeup remover, a makeup, makeup-removing or moisturizing wipes, shaving foams or styling or fixing foams.

Compositions Intended to be Rinsed Off (Rinse-off Compositions)

According to advantageous embodiments, the composition is a skincare and/or haircare composition, preferably for cleansing and/or treating the skin and/or the hair, said composition being in a fluid form. It is advantageously a shower gel, a shampoo, a rinse-off hair conditioner, a skin or hair mask, intended to be rinsed off after use.

For the rinse-off hair conditioners, the composition may advantageously be a relatively viscous formulation, for example a cream, in the form of an emulsion.

Compositions Not Intended to be Rinsed Off (Leave-on Compositions)

According to advantageous embodiments, the composition is a skincare and/or haircare composition, in the form of a fluid or in another form, preferably for treating and/or protecting and/or modifying the appearance of the skin and/or the hair, which is intended to be left on the skin and/or the hair after application.

It may be, for example, a leave-on hair conditioner, a disentangling milk, a disentangling lotion, a smoothing lotion, a cuticle coat, a styling haircare product, a styling and restyling haircare product, an antisun product (antisun cream, antisun milk or antisun oil), a care cream, a makeup remover, a makeup, makeup-removing or moisturizing wipes, shaving foams, styling or fixing foams, or styling or fixing gels.

The composition according to the invention may be a hair dye composition. Such compositions are known to those skilled in the art. It is pointed out that hair dye compositions may consist of several hair dye products intended to be mixed together by the user. In the present patent application, unless otherwise mentioned or specifically stated, the term “hair dye composition” covers either a complete composition or a product intended to be mixed with another by the user. In the present patent application, the term “dyeing the hair” covers any modification of the color of the hair, whether it is a matter of an actual dyeing operation, bleaching, or a combination of bleaching and dyeing.

The hair dye composition may comprise an oxidation base (oxidation dye precursors). It may comprise an oxidizing agent. It may comprise a coupler (coloration modifiers). It may comprise a direct coloring agent (direct dyes). The composition comprises a cosmetically acceptable vector. The composition may also comprise adjuvants.

According to one embodiment, it is a composition for long-lasting dyeing comprising an oxidation base, an oxidizing agent and optionally a coupler, preferably in the form of two products to be combined, a product comprising the oxidation base and a product comprising the oxidizing agent.

According to one embodiment, it is a composition for temporary or long-lasting dyeing comprising a direct dye and optionally an oxidizing agent.

According to one embodiment, it is a composition for bleaching or lightening the hair, comprising an oxidizing agent.

Direct dyes that may be mentioned include neutral, acidic or cationic nitrobenzene dyes, neutral, acidic or cationic azo direct dyes, neutral, acidic or cationic quinone and in particular anthraquinone direct dyes, azine direct dyes, methine direct dyes, tetraazapentamethine direct dyes, triarylmethane direct dyes, indoamine direct dyes and natural direct dyes.

Oxidizing agents that may be mentioned include hydrogen peroxide, urea peroxide, alkali metal bromates, persalts such as perborates and persulfates, peracids and enzymes, especially peroxidases, 2-electron oxidoreductases, and 4-electron oxygenases.

Couplers that may be mentioned include meta-phenylenediamines, meta-aminophenols, meta-diphenols, naphthalene-based couplers and heterocyclic couplers.

As cosmetically acceptable vectors that are preferred in the dye compositions, mention may be made of water and/or mixtures thereof with solvents, for example ethanol, isopropanol, polyols and polyol ethers, for instance 2-butoxyethanol, propylene glycol, and aromatic alcohols, for instance benzyl alcohol or phenoxyethanol.

The adjuvants may be anionic, nonionic, cationic or zwitterionic or amphoteric surfactants, anionic, neutral or cationic polymers, mineral or organic thickeners, antioxidants, penetrants, sequestrants, fragrances, buffers, dispersants, conditioning agents, film-forming agents, ceramides, preserving agents or opacifiers. Needless to say, the ingredients mentioned above may be used as adjuvants in the hair dye compositions.

Preparation of the Composition

The composition may be prepared according to techniques known to those skilled in the art for the hot or cold mixing of ingredients, where appropriate involving prior dissolution or melting. The ingredients (i), (ii), optionally (iii), optionally (iv), the cosmetically acceptable vector, and optionally other ingredients may especially be introduced separately into the composition, or in the form of fluid or nonfluid concentrated ingredients, especially in the form of a fluid concentrated ingredient comprising the ampholytic copolymer and a conditioning agent (iii).

The concentrated ingredient may be in solid or liquid (fluid) form. When it is in liquid form, it may be a solution, a dispersion of solid particles in a liquid phase, or an emulsion or microemulsion comprising an inner liquid phase dispersed in an outer liquid phase. It may especially be an emulsion in an outer aqueous phase. Thus, it may be a concentrated ingredient comprising water, in the form of a direct emulsion comprising droplets of the conditioning agent a) dispersed in water.

In the concentrated ingredient, the weight amount of water is less than 90% by weight and preferably less than 75% by weight. This amount may even be less than 50% by weight, and even zero.

The weight ratio between the ampholytic copolymer (i) and the agent (iii) in the concentrated ingredient is preferably between 0.05 (5/100) and 9 (90/10), preferably between 0.05 and 0.5 (25/50) and preferably between 0.075 and 0.3.

The ingredient advantageously comprises:
from 10% to 75% by weight and preferably from 20% to 70% by weight of agent (iii),
from 0.5% to 20% by weight and preferably from 1% to 15% by weight of ampholytic copolymer (i),
from 0 to 15% by weight of a surfactant, and
optionally water.

It is mentioned that the concentrated ingredient may also comprise an active agent intended to produce an effect on the skin and/or the hair. This may be, for example, an antidandruff agent, a UV-protecting agent or a coloration-protecting agent. These active agents may be organic compounds or mineral particles. If they are included in the concentrated ingredient, the active agents may be dispersed in the conditioning agent a);
as a solution, optionally with a cosolvent for the agent and the active agent,
as a dispersion of solid particles, or
as an emulsion dispersed in the form of droplets in the conditioning agent or in a solution comprising the conditioning agent.

The concentrated ingredient may thus be a multiple emulsion comprising an outer aqueous phase, an intermediate phase, dispersed in the outer phase, comprising the conditioning agent, and an inner phase dispersed in the intermediate phase, comprising the active agent.

The active agent may thus be vectorized by the conditioning agent on the skin and/or the hair.

In the concentrated ingredient, the overall charge of the polymer for aiding deposition, and its solubility or stability, may vary according to the pH. Preferably, the pH is such that the overall charge is positive or neutral.

Other details or advantages of the invention will become apparent in the light of the nonlimiting examples that follow.

### EXAMPLE 1

The products whose compositions are given below are prepared. The starting materials used are identified by the INCI names and/or by the commercial references. The amounts indicated are given as active materials.

Polymer A is a copolymer comprising 33% by number of units derived from DIQUAT and 67% by number of units derived from acrylic acid, with a molecular mass of about 1 000 000.

Shampoo and/or shower gel

| | 1 | 2 | 3 | 4 | 5 | 6 | 7 | 8 |
|---|---|---|---|---|---|---|---|---|
| Sodium Lauryl Ether Sulfate (2 EO) | 14% | 14% | 10% | 10% | 14% | 14% | 10% | 12% |
| Empicol ® ESB-3M (Huntsman) | | | | | | | | |
| Cocamidopropyl Betaine | 2% | 2% | 2% | 2% | 2% | 2% | 2% | 3% |
| Mirataine ® BET C-30 (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.3% | 0.2% | 0.3% | 0.2% | 0.3% | 0.2% | 0.3% |
| Sodium chloride | 0% | 0% | 0% | 0% | 0.1% | 0.5% | 0.1% | 0% |
| Sodium hydroxide | 0.2% | 0.2% | 0.2% | 0.2% | 0.2% | 0.2% | 0.2% | 0.2% |
| Acrylate/C₁₀₋₃₀ Alkyl Acrylate | 1% | 1% | 1% | 1% | — | — | — | — |
| Crosspolymer | | | | | | | | |
| Carbopol ® ETD-2020 (Noveon) | | | | | | | | |
| Acrylate Copolymer | — | — | — | — | 1.5% | 1.5% | 1.5% | — |
| Carbopol ® Aqua SF-1 (Noveon) | | | | | | | | |
| Carbomer | — | — | — | — | — | — | — | 1.2% |
| Carbopol ® 980 (Noveon) | | | | | | | | |
| Glycerol | — | 1% | — | 1% | — | 0.5% | — | 0.5% |
| Glycol Distearate, Laureth-4, | — | — | 1.5% | — | — | 1.5% | — | 1.5% |
| Cocamidopropyl Betaine | | | | | | | | |
| Euperlan ® PK-3000 AM (Cognis) | | | | | | | | |
| Glycol Distearate, Laureth-7, Sodium | — | — | — | 2% | — | — | 2% | — |
| Cocoamphoacetate, Cocamidopropyl | | | | | | | | |
| Betaine, Sodium Laureth Sulfate | | | | | | | | |
| Mirasheen ® CP-820/G (Rhodia) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 9 | 10 | 11 | 12 | 13 | 14 | 15 | 16 |
|---|---|---|---|---|---|---|---|---|
| Sodium Lauryl Ether Sulfate (2 EO) | 14% | 11% | 10% | 10% | 12% | 12% | 10% | 10% |
| Empicol ® ESB-3M (Huntsman) | | | | | | | | |
| Coco Betaine | 2% | — | 3% | — | — | 4% | 3% | — |
| Mirataine ® BB-FLA (Rhodia) | | | | | | | | |
| Disodium Cocoamphodiacetate | — | 3% | — | 3% | 4% | — | — | 2% |
| Miranol ® C2M Conc NP (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | | | 0.5% | 0.5% | | | | 1.8% |
| Sodium hydroxide | — | — | — | — | — | — | 0.5% | — |
| PEG-200 Hydrogenated Glyceryl | 0.7% | 1.2% | — | — | 0.5% | — | — | — |
| Palmate | | | | | | | | |
| Rewoderm ® LI 520-70 (Degussa) | | | | | | | | |
| PEG-150 Distearate | — | — | — | — | — | 1% | 0.4% | — |
| Rewopal ® PEG-6000 DS (Degussa) | | | | | | | | |
| Xanthan gum | — | — | 0.6% | 0.6% | 0.6% | — | — | — |
| Rhodicare ® XC (Rhodia) | | | | | | | | |
| Hydroxyethyl cellulose | — | — | — | — | — | — | 1% | — |
| Natrosol ® 250-HHR HEC (Aqualon) | | | | | | | | |
| Hydroxypropyl Guar | — | — | — | — | — | — | — | 0.8% |
| Jaguar ® HP-105 (Rhodia) | | | | | | | | |
| Propylene Glycol | 0.3% | 0.3% | — | — | — | 0.4% | 0.3% | 0.3% |
| Glycol Distearate, Laureth-4, | 1.5% | — | 1.5% | — | — | 1.5% | — | 1.5% |
| Cocamidopropyl Betaine | | | | | | | | |
| Euperlan ® PK-3000 AM (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 17 | 18 | 19 | 20 | 21 | 22 | 23 | 24 |
|---|---|---|---|---|---|---|---|---|
| Sodium Lauryl Ether Sulfate (2 EO) | 14% | 11% | 10% | 10% | 12% | 12% | 10% | 10% |
| Empicol ® ESB-3M (Huntsman) | | | | | | | | |
| Disodium Cocoamphodiacetate | 2% | 3% | 2% | 3% | 4% | 2% | 2% | 2% |
| Miranol ® C2M Conc NP (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | 0.2% | 0.15% | 0.1% | 0.15% | 0.15% | 0.15% | 0.15% | 0.15% |
| Sodium hydroxide | 0.2% | 0.2% | 0.2% | 0.2% | 0.2% | 0.2% | 0.2% | 0.2% |
| Acrylate/C₁₀₋₃₀ Alkyl Acrylate | 1% | 1% | 1% | 1% | 1% | 1% | 1% | 1% |
| Crosspolymer | | | | | | | | |
| Carbopol ® ETD-2020 (Noveon) | | | | | | | | |
| Guar Hydroxypropyltrimonium | 0.1% | — | — | — | — | — | — | — |
| Chloride | | | | | | | | |
| Jaguar ® C-13S (Rhodia) | | | | | | | | |
| Guar Hydroxypropyltrimonium | — | 0.1% | — | 0.1% | — | — | — | — |
| Chloride | | | | | | | | |
| Jaguar ® Excel (Rhodia) | | | | | | | | |
| Hydroxypropyl Guar | — | — | 0.3% | — | — | — | — | — |
| Hydroxypropyltrimonium Chloride | | | | | | | | |
| Jaguar ® C-162 (Rhodia) | | | | | | | | |
| Polyquaternium-10 | — | — | — | 0.1% | 0.3% | — | — | — |
| Ucare Polymer ® JR-400 (Amerchol) | | | | | | | | |
| Polyquaternium-7 | — | — | — | — | — | 0.3% | — | — |
| Merquat ® 550 (Nalco) | | | | | | | | |
| Polyquaternium-11 | — | — | — | — | — | — | 0.05% | — |
| Mirapol ® PQ-11 (Rhodia) | | | | | | | | |
| Polyquaternium-22 | — | — | — | — | — | — | — | 0.1% |
| Merquat ® 280 (Nalco) | | | | | | | | |
| Glycol Distearate, Laureth-4, | 1.5% | — | — | — | 1.5% | — | 1.5% | 1.5% |
| Cocamidopropyl Betaine | | | | | | | | |
| Euperlan ® PK-3000 AM (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 25 | 26 | 27 | 28 | 29 | 30 | 31 | 32 |
|---|---|---|---|---|---|---|---|---|
| Anionic surfactant: | 14% | 11% | 10% | 10% | 12% | 12% | 10% | 10% |
| Sodium Lauryl Ether Sulfate (2 EO) | | | | | | | | |
| Empicol ® ESB-3M (Huntsman) | | | | | | | | |
| Cocamidopropyl Betaine | 2% | — | 3% | — | — | 4% | 3% | 3% |
| Mirataine ® BET C-30 (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | 0.1% | 0.1% | 0.1% | — | 0.1% | — | — | — |
| Acrylate Copolymer | 1.5% | 1.5% | 1.5% | 1.5% | 1.5% | 1.5% | 1.5% | 1.5% |
| Carbopol ® Aqua SF-1 (Noveon) | | | | | | | | |
| Sodium hydroxide | 0.2% | 0.2% | 0.2% | 0.2% | 0.2% | 0.2% | 0.2% | 0.2% |
| Guar Hydroxypropyltrimonium Chloride | 0.1% | — | — | 0.1% | — | — | — | — |
| Jaguar ® C-13S (Rhodia) | | | | | | | | |
| Polyquaternium-10 | — | 0.3% | — | — | — | — | — | — |
| Ucare Polymer ® JR-400 (Amerchol) | | | | | | | | |
| Polyquaternium-39 | — | — | 0.3% | — | — | — | — | — |
| Merquat ® Plus 3330 (Nalco) | | | | | | | | |
| Polyquaternium-44 | — | — | — | 0.1% | 0.1% | — | — | — |
| Luviquat ® UltraCare (BASF) | | | | | | | | |
| Polyquaternium-67 | — | — | — | — | — | 0.2% | — | — |
| SoftCAT ® Polymer SL (Amerchol) | | | | | | | | |
| Polymethacrylamidopropyltrimonium | — | — | — | — | — | — | 0.2% | |
| Chloride | | | | | | | | |
| Polycare 133 (Rhodia) | | | | | | | | |
| Acrylamidopropyltrimonium Chloride/ | — | — | — | — | — | — | — | 0.3% |
| Acrylamide Copolymer | | | | | | | | |
| Salcare ® SC-60 (Ciba SC) | | | | | | | | |
| Glycol Distearate, Laureth-4, | 1.5% | 1.5% | 1.5% | 1.5% | 1.5% | 1.5% | 1.5% | 1.5% |
| Cocamidopropyl Betaine | | | | | | | | |
| Euperlan ® PK-3000 AM (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 33 | 34 | 35 | 36 | 37 | 38 | 39 | 40 |
|---|---|---|---|---|---|---|---|---|
| Sodium Lauryl Ether Sulfate (2 EO) | — | 14% | 14% | — | 14% | — | — | 14% |
| Empicol ® ESB-3M (Huntsman) | | | | | | | | |
| Ammonium Lauryl Ether Sulfate (2 EO) | 14% | — | — | 14% | — | 14% | 14% | — |
| Rhodapex ® EA-2 (Rhodia) | | | | | | | | |
| Disodium Cocoamphodiacetate | 2% | 1.5% | 1.5% | 2% | 1.5% | 2% | 2% | 1.5% |
| Miranol ® C2M Conc NP (Rhodia) | | | | | | | | |
| 0.6 μm Dimethicone emulsion: | 1% | — | — | — | — | — | — | — |
| Mirasil ® DM-500,000 (Rhodia) | | | | | | | | |
| 0.9 μm Dimethicone emulsion: | — | 1% | — | — | — | — | — | — |
| Mirasil ® DM-500,000 (Rhodia) | | | | | | | | |
| 2 μm Dimethicone emulsion: | — | — | 1% | — | — | — | — | — |
| Mirasil ® DME-2 (Rhodia) | | | | | | | | |
| 30 μm Dimethicone emulsion: | — | — | — | 1% | — | — | — | — |
| Mirasil ® DME-30 (Rhodia) | | | | | | | | |
| Amodimethicone microemulsion: | — | — | — | — | 1% | — | — | — |
| Mirasil ® ADM-E (Rhodia) | | | | | | | | |
| Dimethiconol emulsion: | — | — | — | — | — | 1% | — | — |
| Dow Corning ® 1784 HVF (Dow | | | | | | | | |
| Corning) | | | | | | | | |
| Divinyldimethicone/dimethicone | — | — | — | — | — | — | 1% | — |
| copolymer emulsion: | | | | | | | | |
| Dow Corning ® HMW 2220 (Dow | | | | | | | | |
| Corning) | | | | | | | | |
| PEG/PPG —10/2 Dimethicone: | — | — | — | — | — | — | — | 1% |
| Mirasil ® DMCP-93 (Rhodia) | | | | | | | | |
| Polymer A | 0.2% | 0.2% | 0.2% | 0.2% | 0.2% | 0.2% | 0.2% | 0.2% |
| Sodium hydroxide | 0.2% | 0.2% | 0.2% | 0.2% | — | 0.2% | 0.2% | — |
| Sodium chloride | 0.1% | 0.1% | 0.2% | 0.1% | 1% | 0.3% | 0.1% | 1.8% |
| Acrylate Copolymer | 1.5% | 1.5% | — | 2% | — | — | 1.8% | — |
| Carbopol ® Aqua SF-1 (Noveon) | | | | | | | | |
| Carbomer | — | — | 1.2% | — | — | 1% | — | — |
| Carbopol ® 980 (Noveon) | | | | | | | | |
| Glycol Distearate, Laureth-4, | — | — | — | 1.5% | — | 1.5% | — | 1.5% |
| Cocamidopropyl Betaine | | | | | | | | |
| Euperlan ® PK-3000 AM (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |
| | 41 | 42 | 43 | 44 | 45 | 46 | 47 | 48 |
| Sodium Lauryl Ether Sulfate (2 EO) | — | 10% | 6% | — | 12% | 12% | — | 14% |
| Empicol ® ESB-3M (Huntsman) | | | | | | | | |
| Ammonium Lauryl Sulfate | 5% | — | 6% | 7% | — | — | 5% | — |
| Rhodapon ® L-22 (Rhodia) | | | | | | | | |
| Ammonium Lauryl Ether Sulfate (2 EO) | — | — | — | 7% | — | — | 7% | — |
| Rhodapex ® EA-2 (Rhodia) | | | | | | | | |
| Disodium Cocoamphodiacetate | 3% | — | — | — | — | 3% | — | 1% |
| Miranol ® C2M Conc NP (Rhodia) | | | | | | | | |
| Cocamidopropyl Betaine | — | 4% | — | — | 3% | — | 2% | 2% |
| Mirataine ® BET C-30 (Rhodia) | | | | | | | | |
| Disodium Laureth Sulfosuccinate | 6% | — | 2% | — | 2% | — | — | — |
| Geropon ® SBFA-30 (Rhodia) | | | | | | | | |
| Sodium Lauroyl Glutamate | — | 2% | — | — | — | — | — | 2% |
| Protelan ® AGL-95 (Zschimmer & | | | | | | | | |
| Schwarz) | | | | | | | | |
| Coco Glucoside | 2% | — | — | 1% | — | 2% | — | — |
| Plantacare ® 818 UP (Cognis) | | | | | | | | |
| Cocamide MIPA | 1% | — | 1.5% | — | 1% | — | 1% | 1% |
| Empilan ® CIS (Huntsman) | | | | | | | | |
| Laureth-2 | — | 1% | — | — | — | — | — | — |
| Empilan ® KBE-2 (Huntsman) | | | | | | | | |
| Sodium Lauroyl Sarcosinate | — | — | — | 1% | — | — | 2% | — |
| Protelan ® LS-9011 (Zschimmer & | | | | | | | | |
| Schwarz) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium hydroxide | 0.2% | 0.2% | 0.2% | 0.2% | — | 0.2% | 0.2% | — |
| Sodium chloride | 0.1% | 0.1% | 0.2% | 0.1% | 1.6% | 0.3% | 0.1% | 1.2% |
| Acrylate/C₁₀₋₃₀ Alkyl Acrylate | 1% | 1% | 1% | 1% | — | — | — | — |
| Crosspolymer | | | | | | | | |
| Carbopol ® ETD-2020 (Noveon) | | | | | | | | |
| Acrylate Copolymer | — | — | — | — | 1.5% | 1.5% | 1.5% | 1.5% |
| Carbopol ® Aqua SF-1 (Noveon) | | | | | | | | |
| Glycol Distearate, Laureth-4, | — | — | — | 1.5% | — | 1.5% | — | 1.5% |
| Cocamidopropyl Betaine | | | | | | | | |
| Euperlan ® PK-3000 AM (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |
| | 49 | 50 | 51 | 52 | 53 | 54 | 55 | 56 |
| Sodium Lauryl Ether Sulfate (2 EO) | — | 14% | 14% | — | 14% | — | — | 14% |
| Empicol ® ESB-3M (Huntsman) | | | | | | | | |
| Ammonium Lauryl Ether Sulfate (2 EO) | 14% | — | — | 14% | — | 14% | 14% | — |
| Rhodapex ® EA-2 (Rhodia) | | | | | | | | |
| Disodium Cocoamphodiacetate | 2% | 1.5% | 1.5% | 2% | 1.5% | 2% | 2% | 1.5% |
| Miranol ® C2M Conc NP (Rhodia) | | | | | | | | |
| Cocamide MIPA | 1% | 1% | 1% | 1% | 1% | 1% | 1% | 1% |
| Empilan ® CIS (Huntsman) | | | | | | | | |
| Cetrimonium Bromide | 1% | — | — | — | — | — | — | — |
| Rhodaquat ® M-242B/99 (Rhodia) | | | | | | | | |
| Zinc Pyrithione | — | 1% | — | — | — | — | — | — |
| Zinc Omadine ® (Arch Chemical) | | | | | | | | |
| Piroctone Olamine | — | — | 1% | — | — | — | — | — |
| Octopirox ® (Clariant) | | | | | | | | |
| Selenium sulfide | — | — | — | 1% | — | — | — | — |
| Salicylic acid (Rhodia) | — | — | — | — | 1% | — | — | — |
| Benzophenone-3 | — | — | — | — | — | 1% | — | — |
| Uvinul ® M-40 (BASF) | | | | | | | | |
| Ethylhexyl Methoxycinnamate | — | — | — | — | — | — | 1% | — |
| Parsol ® MCX (DSM) | | | | | | | | |
| Polysilicone-15 | — | — | — | — | — | — | — | 1% |
| Parsol ® SLX (DSM) | | | | | | | | |
| Polymer A | 0.2% | 0.2% | 0.2% | 0.2% | 0.2% | 0.2% | 0.2% | 0.2% |
| Sodium hydroxide | — | 0.1% | 0.1% | 0.1% | 0.1% | 0.1% | 0.1% | 0.1% |
| Sodium chloride | 1.1% | 0.1% | 0.2% | 0.1% | 0.2% | 0.3% | 0.1% | 0.1% |
| Acrylate Copolymer | — | 0.8% | 0.7% | 0.7% | 0.7% | — | — | — |
| Carbopol ® Aqua SF-1 (Noveon) | | | | | | | | |
| Carbomer | — | — | — | — | — | 0.5% | 0.5% | 0.5% |
| Carbopol ® 980 (Noveon) | | | | | | | | |
| Glycol Distearate, Laureth-4, | — | — | — | 1.5% | — | 1.5% | — | 1.5% |
| Cocamidopropyl Betaine | | | | | | | | |
| Euperlan ® PK-3000 AM (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 57 | 58 | 59 | 60 | 61 |
|---|---|---|---|---|---|
| Sodium Lauryl Ether Sulfate (2 EO) | 10% | 10% | 14% | 10% | 12% |
| Empicol ® ESB-3M (Huntsman) | | | | | |
| Cocamidopropyl Betaine | 2% | 2% | 2% | 2% | 3% |
| Mirataine ® BET C-30 (Rhodia) | | | | | |
| 0.6 μm Dimethicone emulsion: | 1% | 1% | 1% | 1% | 1% |
| Mirasil ® DM-500,000 (Rhodia) | | | | | |
| Polymer A | 0.2% | 0.3% | 0.3% | 0.2% | 0.3% |
| Sodium chloride | 0% | 0% | 0.5% | 0.1% | 0% |
| Sodium hydroxide | 0.2% | 0.2% | 0.2% | 0.2% | 0.2% |
| Acrylate/C₁₀₋₃₀ Alkyl Acrylate | 1% | 1% | — | — | — |
| Crosspolymer | | | | | |
| Carbopol ® ETD-2020 (Noveon) | | | | | |
| Acrylate Copolymer | — | — | 1.5% | 1.5% | — |
| Carbopol ® Aqua SF-1 (Noveon) | | | | | |
| Carbomer | — | — | — | — | 1.2% |
| Carbopol ® 980 (Noveon) | | | | | |
| Glycerol | — | 1% | 0.5% | — | 0.5% |
| Sodium Laureth Sulfate, Glycol | 1.5% | — | 1.5% | — | 1.5% |
| Distearate Cocamide MEA, Laureth- | | | | | |
| 10 | | | | | |
| Euperlan PK-771 BENZ (Cognis) | | | | | |
| PEG-3 Distearate | — | 1.5% | — | 1.5% | — |
| Genapol TS (Clariant) | | | | | |
| Citric acid | qs | qs | qs | qs | qs |
| Fragrance, preserving agents | qs | qs | qs | qs | qs |
| Demineralized water | qsp | qsp | qsp | qsp | qsp |
| | 100% | 100% | 100% | 100% | 100% |

| | 62 | 63 | 64 | 65 | 66 | 67 | 68 | 69 |
|---|---|---|---|---|---|---|---|---|
| Sodium Lauryl Ether Sulfate (2 EO) | 14% | 10% | 12% | 10% | 14% | 10% | 12% | 10% |
| Empicol ® ESB-3M (Huntsman) | | | | | | | | |
| Coco Betaine | 2% | 3% | 4% | — | 2% | 3% | 4% | — |
| Mirataine ® BB-FLA (Rhodia) | | | | | | | | |
| Disodium Cocoamphodiacetate | — | — | — | 2% | — | — | — | 2% |
| Miranol ® C2M Conc NP (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | | 0.5% | | 1.8% | | 0.5% | | 1.8% |
| Sodium hydroxide | — | — | — | — | — | — | — | — |
| PEG-200 Hydrogenated Glyceryl | 0.7% | — | — | — | 0.7% | — | — | — |
| Palmate | | | | | | | | |
| Rewoderm ® LI 520-70 (Degussa) | | | | | | | | |
| PEG-150 Distearate | — | — | 1% | — | — | — | 1% | — |
| Rewopal ® PEG-6000 DS (Degussa) | | | | | | | | |
| Xanthan gum | — | 0.6% | — | — | — | 0.6% | — | — |
| Rhodicare ® XC (Rhodia) | | | | | | | | |
| Hydroxyethyl cellulose | — | — | — | — | — | — | — | — |
| Natrosol ® 250-HHR HEC (Aqualon) | | | | | | | | |
| Hydroxypropyl Guar | — | — | — | 0.8% | — | — | — | 0.8% |
| Jaguar ® HP-105 (Rhodia) | | | | | | | | |
| Propylene Glycol | 0.3% | — | 0.4% | 0.3% | 0.3% | — | 0.4% | 0.3% |
| Sodium Laureth Sulfate, Glycol | 1.0% | 1.0% | 1.0% | 1.0% | — | — | — | — |
| Distearate Cocamide MEA, Laureth- | | | | | | | | |
| 10 | | | | | | | | |
| Euperlan PK-771 BENZ (Cognis) | | | | | | | | |
| PEG-3 Distearate | — | — | — | — | 1.5% | 1.5% | 1.5% | 1.5% |
| Genapol TS (Clariant) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 70 | 71 | 72 | 73 | 74 | 75 | 76 | 77 |
|---|---|---|---|---|---|---|---|---|
| Sodium Lauryl Ether Sulfate (2 | 14% | 12% | 10% | 10% | 14% | 12% | 10% | 10% |
| EO) | | | | | | | | |
| Empicol ® ESB-3M (Huntsman) | | | | | | | | |
| Disodium Cocoamphodiacetate | 2% | 4% | 2% | 2% | 2% | 4% | 2% | 2% |
| Miranol ® C2M Conc NP | | | | | | | | |
| (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | 0.2% | 0.15% | 0.15% | 0.15% | 0.2% | 0.15% | 0.15% | 0.15% |
| Sodium hydroxide | 0.2% | 0.2% | 0.2% | 0.2% | 0.2% | 0.2% | 0.2% | 0.2% |
| Acrylate/C₁₀₋₃₀ Alkyl Acrylate | 1% | 1% | 1% | 1% | 1% | 1% | 1% | 1% |
| Crosspolymer | | | | | | | | |
| Carbopol ® ETD-2020 (Noveon) | | | | | | | | |
| Guar Hydroxypropyltrimonium | 0.1% | — | — | — | 0.1% | — | — | — |
| Chloride | | | | | | | | |
| Jaguar ® C-13S (Rhodia) | | | | | | | | |
| Guar Hydroxypropyltrimonium | — | — | — | — | — | — | — | — |
| Chloride | | | | | | | | |
| Jaguar ® Excel (Rhodia) | | | | | | | | |
| Hydroxypropyl Guar | — | — | — | — | — | — | — | — |
| Hydroxypropyltrimonium | | | | | | | | |
| Chloride | | | | | | | | |
| Jaguar ® C-162 (Rhodia) | | | | | | | | |
| Polyquaternium-10 | — | 0.3% | — | — | — | 0.3% | — | — |
| Ucare Polymer ® JR-400 | | | | | | | | |
| (Amerchol) | | | | | | | | |
| Polyquaternium-7 | — | — | — | — | — | — | — | — |
| Merquat ® 550 (Nalco) | | | | | | | | |
| Polyquaternium-11 | — | — | 0.05% | — | — | — | 0.05% | — |
| Mirapol ® PQ-11 (Rhodia) | | | | | | | | |
| Polyquaternium-22 | — | — | — | 0.1% | — | — | — | 0.1% |
| Merquat ® 280 (Nalco) | | | | | | | | |
| Sodium Laureth Sulfate, Glycol | 1.5% | 1.5% | 1.5% | 1.5% | — | — | — | — |
| Distearate Cocamide MEA, | | | | | | | | |
| Laureth-10 | | | | | | | | |
| Euperlan PK-771 BENZ (Cognis) | | | | | | | | |
| PEG-3 Distearate | — | — | — | — | 1.5% | 1.5% | 1.5% | 1.5% |
| Genapol TS (Clariant) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 78 | 79 | 80 | 81 | 82 | 83 | 84 | 85 |
|---|---|---|---|---|---|---|---|---|
| Anionic surfactant: | 14% | 11% | 10% | 10% | 12% | 12% | 10% | 10% |
| Sodium Lauryl Ether Sulfate (2 EO) | | | | | | | | |
| Empicol ® ESB-3M (Huntsman) | | | | | | | | |
| Cocamidopropyl Betaine | 2% | — | 3% | — | — | 4% | 3% | 3% |
| Mirataine ® BET C-30 (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | 0.1% | 0.1% | 0.1% | — | 0.1% | — | — | — |
| Acrylate Copolymer | 1.5% | 1.5% | 1.5% | 1.5% | 1.5% | 1.5% | 1.5% | 1.5% |
| Carbopol ® Aqua SF-1 (Noveon) | | | | | | | | |
| Sodium hydroxide | 0.2% | 0.2% | 0.2% | 0.2% | 0.2% | 0.2% | 0.2% | 0.2% |
| Guar Hydroxypropyltrimonium Chloride | 0.1% | — | — | 0.1% | — | — | — | — |
| Jaguar ® C-13S (Rhodia) | | | | | | | | |
| Polyquaternium-10 | — | 0.3% | — | — | — | — | — | — |
| Ucare Polymer ® JR-400 (Amerchol) | | | | | | | | |
| Polyquaternium-39 | — | — | 0.3% | — | — | — | — | — |
| Merquat ® Plus 3330 (Nalco) | | | | | | | | |
| Polyquaternium-44 | — | — | — | 0.1% | 0.1% | — | — | — |
| Luviquat ® UltraCare (BASF) | | | | | | | | |
| Polyquaternium-67 | — | — | — | — | — | 0.2% | — | — |
| SoftCAT ® Polymer SL (Amerchol) | | | | | | | | |
| Polymethacrylamidopropyltrimonium | — | — | — | — | — | — | 0.2% | |
| Chloride | | | | | | | | |
| Polycare 133 (Rhodia) | | | | | | | | |
| Acrylamidopropyltrimonium Chloride/ | — | — | — | — | — | — | — | 0.3% |
| Acrylamide Copolymer | | | | | | | | |
| Salcare ® SC-60 (Ciba SC) | | | | | | | | |
| Sodium Laureth Sulfate, Glycol | 1.5% | 1.5% | 1.5% | 1.5% | 1.5% | 1.5% | 1.5% | 1.5% |
| distearate Cocamide MEA, Laureth-10 | | | | | | | | |
| Euperlan PK-771 BENZ (Cognis) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |
| | 86 | 87 | 88 | 89 | 90 | 91 | 92 | 93 |
| Anionic surfactant: | 14% | 11% | 10% | 10% | 12% | 12% | 10% | 10% |
| Sodium Lauryl Ether Sulfate (2 EO) | | | | | | | | |
| Empicol ® ESB-3M (Huntsman) | | | | | | | | |
| Cocamidopropyl Betaine | 2% | — | 3% | — | — | 4% | 3% | 3% |
| Mirataine ® BET C-30 (Rhodia) | | | | | | | | |
| 0.6 μm Dimethicone emulsion: | 1% | 1% | 1% | 1% | 1% | 1% | 1% | 1% |
| Mirasil ® DM-500,000 (Rhodia) | | | | | | | | |
| Polymer A | 0.2% | 0.2% | 0.2% | 0.2% | 0.2% | 0.2% | 0.2% | 0.2% |
| Sodium chloride | 0.1% | 0.1% | 0.1% | — | 0.1% | — | — | — |
| Acrylate Copolymer | 1.5% | 1.5% | 1.5% | 1.5% | 1.5% | 1.5% | 1.5% | 1.5% |
| Carbopol ® Aqua SF-1 (Noveon) | | | | | | | | |
| Sodium hydroxide | 0.2% | 0.2% | 0.2% | 0.2% | 0.2% | 0.2% | 0.2% | 0.2% |
| Guar Hydroxypropyltrimonium Chloride | 0.1% | — | — | 0.1% | — | — | — | — |
| Jaguar ® C-13S (Rhodia) | | | | | | | | |
| Polyquaternium-10 | — | 0.3% | — | — | — | — | — | — |
| Ucare Polymer ® JR-400 (Amerchol) | | | | | | | | |
| Polyquaternium-39 | — | — | 0.3% | — | — | — | — | — |
| Merquat ® Plus 3330 (Nalco) | | | | | | | | |
| Polyquaternium-44 | — | — | — | 0.1% | 0.1% | — | — | — |
| Luviquat ® UltraCare (BASF) | | | | | | | | |
| Polyquaternium-67 | — | — | — | — | — | 0.2% | — | — |
| SoftCAT ® Polymer SL (Amerchol) | | | | | | | | |
| Polymethacrylamidopropyltrimoium | — | — | — | — | — | — | 0.2% | — |
| Chloride | | | | | | | | |
| Polycare 133 (Rhodia) | | | | | | | | |
| Acrylamidopropyltrimonium Chloride/ | — | — | — | — | — | — | — | 0.3% |
| Acrylamide Copolymer | | | | | | | | |
| Salcare ® SC-60 (Ciba SC) | | | | | | | | |
| PEG-3 Distearate | 1.5% | 1.5% | 1.5% | 1.5% | 1.5% | 1.5% | 1.5% | 1.5% |
| Genapol TS (Clariant) | | | | | | | | |
| Citric acid | qs | qs | qs | qs | qs | qs | qs | qs |
| Fragrance, preserving agents | qs | qs | qs | qs | qs | qs | qs | qs |
| Demineralized water | qsp | qsp | qsp | qsp | qsp | qsp | qsp | qsp |
| | 100% | 100% | 100% | 100% | 100% | 100% | 100% | 100% |

| | 94 | 95 | 96 | 97 | 98 | 99 |
|---|---|---|---|---|---|---|
| Sodium Lauryl Ether Sulfate (2 EO) | — | — | 14% | — | — | 14% |
| Empicol ® ESB-3M (Huntsman) | | | | | | |
| Ammonium Lauryl Ether Sulfate (2 EO) | 14% | 14% | — | 14% | 14% | — |
| Rhodapex ® EA-2 (Rhodia) | | | | | | |
| Disodium Cocoamphodiacetate | 2% | 2% | 1.5% | 2% | 2% | 1.5% |
| Miranol ® C2M Conc NP (Rhodia) | | | | | | |
| 0.6 μm Dimethicone emulsion: | — | — | — | — | — | — |
| Mirasil ® DM-500,000 (Rhodia) | | | | | | |
| 0.9 μm Dimethicone emulsion: | — | — | — | — | — | — |
| Mirasil ® DM-500,000 (Rhodia) | | | | | | |
| 2 μm Dimethicone emulsion: | — | — | — | — | — | — |
| Mirasil ® DME-2 (Rhodia) | | | | | | |
| 30 μm Dimethicone emulsion: | 1% | — | — | 1% | — | — |
| Mirasil ® DME-30 (Rhodia) | | | | | | |
| Amodimethicone microemulsion: | — | — | — | — | — | — |
| Mirasil ® ADM-E (Rhodia) | | | | | | |
| Dimethiconol emulsion: | — | 1% | — | — | 1% | — |
| Dow Corning ® 1784 HVF (Dow | | | | | | |
| Corning) | | | | | | |
| Divinyl dimethicone/dimethicone | — | — | — | — | — | — |
| copolymer emulsion: | | | | | | |
| Dow Corning ® HMW 2220 (Dow | | | | | | |
| Corning) | | | | | | |
| PEG/PPG —10/2 Dimethicone: | — | — | 1% | — | — | 1% |
| Mirasil ® DMCP-93 (Rhodia) | | | | | | |
| AA-DIQUAT (Rhodia) | 0.2% | 0.2% | 0.2% | 0.2% | 0.2% | 0.2% |
| Sodium hydroxide | 0.2% | 0.2% | — | 0.2% | 0.2% | — |
| Sodium chloride | 0.1% | 0.3% | 1.8% | 0.1% | 0.3% | 1.8% |
| Acrylate Copolymer | 2% | — | — | 2% | — | — |
| Carbopol ® Aqua SF-1 (Noveon) | | | | | | |
| Carbomer | — | 1% | — | — | 1% | — |
| Carbopol ® 980 (Noveon) | | | | | | |
| Sodium Laureth Sulfate, Glycol | 1.5% | 1.5% | 1.5% | — | — | — |
| Distearate Cocamide MEA, Laureth-10 | | | | | | |
| Euperlan PK-771 BENZ (Cognis) | | | | | | |
| PEG-3 Distearate | — | — | — | 1.0% | 1.5% | 1.5% |
| Genapol TS (Clariant) | | | | | | |
| Citric acid | qs | qs | qs | qs | qs | qs |
| Fragrance, preserving agents | qs | qs | qs | qs | qs | qs |
| Demineralized water | qsp | qsp | qsp | qsp | qsp | qsp |
| | 100% | 100% | 100% | 100% | 100% | 100% |

| | 100 | 101 | 102 | 103 | 104 | 105 |
|---|---|---|---|---|---|---|
| Sodium Lauryl Ether Sulfate (2 EO) | — | 12% | 14% | — | 12% | 14% |
| Empicol ® ESB-3M (Huntsman) | | | | | | |
| Ammonium Lauryl Sulfate | 7% | — | — | 7% | — | — |
| Rhodapon ® L-22 (Rhodia) | | | | | | |
| Ammonium Lauryl Ether Sulfate (2 EO) | 7% | — | — | 7% | — | — |
| Rhodapex ® EA-2 (Rhodia) | | | | | | |
| Disodium Cocoamphodiacetate | — | 3% | 1% | — | 3% | 1% |
| Miranol ® C2M Conc NP (Rhodia) | | | | | | |
| Cocamidopropyl Betaine | — | — | 2% | — | — | 2% |
| Mirataine ® BET C-30 (Rhodia) | | | | | | |
| Disodium Laureth Sulfosuccinate | — | — | — | — | — | — |
| Geropon ® SBFA-30 (Rhodia) | | | | | | |
| Sodium Lauroyl Glutamate | — | — | 2% | — | — | 2% |
| Protelan ® AGL-95 (Zschimmer & | | | | | | |
| Schwarz) | | | | | | |
| Coco Glucoside | 1% | 2% | — | 1% | 2% | — |
| Plantacare ® 818 UP (Cognis) | | | | | | |
| Cocamide MIPA | — | — | 1% | — | — | 1% |
| Empilan ® CIS Huntsman | | | | | | |
| Laureth-2 | — | — | — | — | — | — |
| Empilan ® KBE-2 (Huntsman) | | | | | | |
| Sodium Lauroyl Sarcosinate | 1% | — | — | 1% | — | — |
| Protelan ® LS-9011 (Zschimmer & | | | | | | |
| Schwarz | | | | | | |
| 0.6 μm Dimethicone emulsion: | 1% | 1% | 1% | 1% | 1% | 1% |
| Mirasil ® DM-500,000 (Rhodia) | | | | | | |
| Polymer A | 0.2% | 0.2% | 0.2% | 0.2% | 0.2% | 0.2% |
| Sodium hydroxide | 0.2% | 0.2% | — | 0.2% | 0.2% | — |
| Sodium chloride | 0.1% | 0.3% | 1.2% | 0.1% | 0.3% | 1.2% |
| Acrylate/C₁₀₋₃₀ Alkyl Acrylate Crosspolymer | 1% | — | — | 1% | — | — |
| Carbopol ® ETD-2020 (Noveon) | | | | | | |
| Acrylate Copolymer | — | 1.5% | 1.5% | — | 1.5% | 1.5% |
| Carbopol ® Aqua SF-1 (Noveon) | | | | | | |
| Sodium Laureth Sulfate, Glycol Distearate | 1.5% | 1.5% | 1.5% | — | — | — |
| Cocamide MEA, Laureth-10 | | | | | | |
| Euperlan PK-771 BENZ (Cognis) | | | | | | |
| PEG-3 Distearate | — | — | — | 1.5% | 1.5% | 1.5% |
| Genapol TS (Clariant) | | | | | | |
| Citric acid | qs | qs | qs | qs | qs | qs |
| Fragrance, preserving agents | qs | qs | qs | qs | qs | qs |
| Demineralized water | qsp | qsp | qsp | qsp | qsp | qsp |
| | 100% | 100% | 100% | 100% | 100% | 100% |

| | 106 | 107 | 108 | 109 | 110 | 111 |
|---|---|---|---|---|---|---|
| Sodium Lauryl Ether Sulfate (2 EO) | — | — | 14% | — | — | 14% |
| Empicol ® ESB-3M (Huntsman) | | | | | | |
| Ammonium Lauryl Ether Sulfate (2 EO) | 14% | 14% | — | 14% | 14% | — |
| Rhodapex ® EA-2 (Rhodia) | | | | | | |
| Disodium Cocoamphodiacetate | 2% | 2% | 1.5% | 2% | 2% | 1.5% |
| Miranol ® C2M Conc NP (Rhodia) | | | | | | |
| Cocamide MIPA | 1% | 1% | 1% | 1% | 1% | 1% |
| Empilan ® CIS (Huntsman) | | | | | | |
| Cetrimonium bromide | — | — | — | — | — | — |
| Rhodaquat ® M-242B/99 (Rhodia) | | | | | | |
| Zinc Pyrithione | — | — | — | — | — | — |
| Zinc Omadine ® (Arch Chemical) | | | | | | |
| Piroctone Olamine | — | — | — | — | — | — |
| Octopirox ® (Clariant) | | | | | | |
| Selenium sulfide | 1% | — | — | 1% | — | — |
| Salicylic acid (Rhodia) | — | — | — | — | — | — |
| Benzophenone-3 | — | 1% | — | — | 1% | — |
| Uvinul ® M-40 (BASF) | | | | | | |
| Ethylhexyl Methoxycinnamate | — | — | — | — | — | — |
| Parsol ® MCX (DSM) | | | | | | |
| Polysilicone-15 | — | — | 1% | — | — | 1% |
| Parsol ® SLX (DSM) | | | | | | |
| Polymer A | 0.2% | 0.2% | 0.2% | 0.2% | 0.2% | 0.2% |
| Sodium hydroxide | 0.1% | 0.1% | 0.1% | 0.1% | 0.1% | 0.1% |
| Sodium chloride | 0.1% | 0.3% | 0.1% | 0.1% | 0.3% | 0.1% |
| Acrylate Copolymer | 0.7% | — | — | 0.7% | — | — |
| Carbopol ® Aqua SF-1 (Noveon) | | | | | | |
| Carbomer | — | 0.5% | 0.5% | — | 0.5% | 0.5% |
| Carbopol ® 980 (Noveon) | | | | | | |
| Sodium Laureth Sulfate, Glycol | 1.5% | 1.5% | 1.5% | — | — | — |
| Disterate Cocamide MEA, Laureth-10 | | | | | | |
| Euperlan PK-771 BENZ (Cognis) | | | | | | |
| PEG-3 Distearate | — | — | — | 1.5% | 1.5% | 1.5% |
| Genapol TS (Clariant) | | | | | | |
| Citric acid | qs | qs | qs | qs | qs | qs |
| Fragrance, preserving agents | qs | qs | qs | qs | qs | qs |
| Demineralized water | qsp | qsp | qsp | qsp | qsp | qsp |
| | 100% | 100% | 100% | 100% | 100% | 100% |

Styling gels

| | 112 | 113 | 114 | 115 | 116 | 117 |
|---|---|---|---|---|---|---|
| Polymer A | 2% | 1.5% | 0.1% | 2% | 2% | 1.5% |
| Hydroxypropyl Guar | 1.5% | | | | 1.5% | |
| Jaguar ® HP60 (Rhodia) | | | | | | |
| Glycerol | 1% | | | 1% | 1% | |
| Aminomethylpropanol AMP | | | 0.16% | | | |
| Hydroxyethylcellulose | | 0.75% | | | | 0.75% |
| Natrosol ® 250 HP (Hercules) | | | | | | |
| Propylene Glycol | | 1% | 3% | 0.5% | | 1% |
| Panthenol | | 0.05% | | | | 0.05% |
| D-Panthenol USP (BASF) | | | | | | |
| Carbomer | | | 0.2% | | | |
| Carbomer C (Rhodia) | | | | | | |
| Glucose | | | 7% | | | |
| Hydroxypropyl Guar | | | | 1.5% | | |
| Jaguar ® HP105 (Rhodia) | | | | | | |
| Polysorbate 20 | | | | | | 0.1% |
| Alkamuls ® T-20C (Rhodia) | | | | | | |
| Fragrance, preserving agents | qs | qs | qs | qs | qs | qs |
| Water | qsp | qsp | qsp | qsp | qsp | qsp |

| | 118 |
|---|---|
| Polymer A | 5% |
| Oleth 20 | 0.5% |
| Rhodiasurf ® ON-870 (Rhodia) | |
| Simethicone | 0.3% |
| Mirasil ® SM (Rhodia) | |
| Acrylates/Steareth-20 Methacrylates | 3.33% |
| Copolymer | |
| Aculyn ® 22 (Rohm & Haas) | |
| Panthenol | 0.3% |
| D-Panthenol USP (BASF) | |
| Glycerol | 0.5% |
| NaOH 10% | 2% |
| Fragrance, preserving agents | qs |
| Water | qsp |

Coloring shampoo

| | 119 | 120 | 121 | 122 | 123 | 124 |
|---|---|---|---|---|---|---|
| Basic Red 51 | 1% | 1% | 1% | 1% | 1% | 1% |
| 306008 Arianor ® Cherry Red (LCW) | | | | | | |
| Polysilicone 15 | | | 1% | 1% | | |
| Parsol ® SLX (DSM) | | | | | | |
| Octyl Methoxycinnamate | | | | | 1% | 1% |
| Eusolex ® 2292 (Merck) | | | | | | |
| Disodium Cocoamphodipropionate | 3.15% | 3.15% | 3.15% | 3.15% | 3.15% | 3.15% |
| Miranol ® C2M-SF (Rhodia) | | | | | | |
| Lauramine Oxide | 4.2% | | 4.2% | | 4.2% | |
| Rhodamox ® LO (Rhodia) | | | | | | |
| Trideceth Carboxamide MEA | 3% | | 3% | | 3% | |
| Amideth ® A-15 (Kao) | | | | | | |
| Acrylates/Aminoacrylates/C10-30 Alkyl | 2.5% | 2.5% | 2.5% | 2.5% | 2.5% | 2.5% |
| PEG-20 Itaconate Copolymer | | | | | | |
| Structure ® PLUS (National Starch) | | | | | | |
| AA-Diquat (Rhodia) | 0.5% | 0.5% | 0.5% | 0.5% | 0.5% | 0.5% |
| Lauramine Oxide | | 4.2% | | 4.2% | | 4.2% |
| Incromine ® Oxide L (Croda) | | | | | | |
| PPG-2 Hydroxyethylcocamide | | 3% | | 3% | | 3% |
| Promidium ® CO (Uniqema) | | | | | | |
| Fragrances, preserving agents | qs | qs | qs | qs | qs | qs |
| Water | qsp | qsp | qsp | qsp | qsp | qsp |

| | 125 | 126 |
|---|---|---|
| Basic Red 51 | 1% | 1% |
| 306008 Arianor ® Cherry Red (LCW) | | |
| Benzophenone-4 | 1% | 1% |
| Uvinul ® MS40 (BASF) | | |
| Disodium Cocoamphodipropionate | 3.15% | 3.15% |
| Miranol ® C2M-SF (Rhodia) | | |
| Lauramine Oxide | 4.2% | |
| Rhodamox ® LO (Rhodia) | | |
| Trideceth Carboxamide MEA | 3% | |
| Amideth ® A-15 (Kao) | | |
| Acrylates/Aminoacrylates/C10-30 Alkyl | 2.5% | 2.5% |
| PEG-20 Itaconate Copolymer | | |
| Structure ® PLUS (National Starch) | | |
| Polymer A | 0.5% | 0.5% |
| Lauramine Oxide | | 4.2% |
| Incromine ® Oxide L (Croda) | | |
| PPG-2 Hydroxyethylcocamide | | 3% |
| Promidium ® CO (Uniqema) | | |
| Fragrances, preserving agents | qs | qs |
| Water | qsp | qsp |

Hair conditioners

| | 127 | 128 | 129 | 130 |
|---|---|---|---|---|
| Polymer A | 0.3% | 0.3% | 0.5% | 0.5% |
| SDA Alcohol 40 (Prolabo) | 40% | 40% | 40% | 40% |
| Polyquaternium-2 | 1.5% | 1.5% | | |
| Mirapol ® A-15 (Rhodia) | | | | |
| PEG/PPG-22/24 Dimethicone | 1.0% | 1.0% | 1.0% | 1.0% |
| Mirasil ® DMCO (Rhodia) | | | | |
| Benzophenone-4 | 0.5% | | | 0.3% |
| Uvinul ® MS40 (BASF) | | | | |
| Hydrolyzed Keratin | 0.2% | 0.2% | 0.2% | 0.2% |
| Dimethicone | | | 1% | 1% |
| Mirasil ® DM500,000 (Rhodia) | | | | |
| Dye | | | qs | Qs |
| Fragrances, preserving agents | qs | qs | qs | Qs |
| Water | qsp | qsp | qsp | Qsp |

| | 131 | 132 | 133 | 134 | 135 |
|---|---|---|---|---|---|
| Glyceryl Stearate (and) PEG-100 Stearate | 7% | 7% | 4% | 4% | 4% |
| Polymer A | 0.2% | 0.2% | 0.2% | 0.2% | 0.2% |
| Cetearyl Alcohol | 6% | 6% | | | |
| Wheat (Triticum vulgare) Germ Oil | 5% | 5% | 4% | 4% | 4% |
| Coconut (Cocos nucifera) Oil | 5% | 5% | | | |
| Mineral oil | 7% | 7% | | | |
| Marcol ® 82 (Mobil) | | | | | |
| Dimethicone (and) Laureth-8 (and) Succinoglycan | 4% | | | | |
| Mirasil ® DME-30 (Rhodia) | | | | | |
| Dimethicone (and) Laureth-7 | | 4% | | | |
| Hydroxypropyl trimonium Hydrolyzed Wheat | 0.5% | 0.5% | | | |
| Protein | | | | | |
| Hydroxypropyl Guar | | | 0.5% | | 0.5% |
| Jaguar ® HP-8 Rhodia | | | | | |
| Propylene glycol | | | 2% | 2% | 2% |
| Cetearyl Octanoate | | | 2% | 2% | 2% |
| Cetyl Alcohol | | | 5% | 5% | 5% |
| Lauryldimonium Hydroxypropyl Hydrolyzed | | | 0.5 | 0.5 | 0.5 |
| Keratin | | | | | |
| Polyquaternium-2 | | | 2% | 2% | |
| Mirapol ® A-15 (Rhodia) | | | | | |
| Fragrance, preserving agents | qs | qs | qs | qs | qs |
| Water | qsp | qsp | qsp | qsp | qsp |

| | 134 | 135 | 136 | 137 | 138 |
|---|---|---|---|---|---|
| Glyceryl Stearate (and) PEG-100 Stearate | 0.3% | 0.3% | 0.3% | 0.3% | 0.3% |
| Polymer A | 0.2% | 0.2% | 0.2% | 0.2% | 0.2% |
| Polysorbate-60 | 0.3% | | 0.3% | 0.3% | 0.3% |
| Alkamuls T-80C (Rhodia) | | | | | |
| Silicone (Dimethicone) (and) laureth-7 or laureth-8 | 6% | 8% | 6% | 6% | 6% |
| Hydroxypropyl Guar | | | | 0.2% | 0.2% |
| Jaguar ® HP-8 (Rhodia) | | | | | |
| Stearyl Alcohol | 0.2% | 0.2% | 0.2% | 0.2% | 0.2% |
| Cetyl Alcohol | 0.3% | 0.3% | 0.3% | 0.3% | 0.3% |
| Quaternium-18 | 0.75% | 0.75% | | 0.75% | |
| Hydroxyethyl cellulose | 0.5% | 0.5% | 0.5% | 0.5% | 0.5% |
| Natrosol ® 250-HHR HEC (Aqualon) | | | | | |
| Fragrance, preserving agents | qs | qs | qs | qs | qs |
| Water | qsp | qsp | qsp | qsp | qsp |

### EXAMPLE 2

This example illustrates the preparation of a shampoo.

Polymer A is a copolymer comprising 33% by number of units derived from DIQUAT and 67% by number of units derived from acrylic acid, with a molecular mass of about 1 000 000.

Composition (Active Material):

14% SLES
2% CAPB
0.1% NaCl
0.2% Polymer A
1% DM
1.5% Carbopol SF1
   Starting Materials Used:
   Commercial name Carbopol Aqua SF1, INCI name Acrylates copolymer, 30% AM, supplier Noveon
   SLES: commercial name Empicol ESB 3M, INCI name Sodium Lauryl Ether Sulfate, 27% AM, supplier Huntsman
   CAPB: commercial name Mirataine BET C30, INCI name Cocamidopropylbetaine, 30% AM, supplier Rhodia
   DM: Emulsion of Mirasil DM 500 000 at 0.6 μm, INCI name: Dimethicone, 65% AM
   Polymer A
   Kathon CG, supplier SEPPIC
   NaCl, supplier Prolabo
      The masses of the starting materials are weighed out on a balance with a precision of 0.01 g and/or on a 0.001 g precision balance.

A. Preparation Phase 1

1/ Preparation of the Water-thickener Mixture in a Beaker

weigh out the mass of Carbopol Aqua SF1:5.0 g
weigh out and add to the liquid solution 10 g of distilled water mix with a frame paddle at 150 rpm for 5 minutes.
   2/ Addition of the Anionic Surfactant
weigh out the mass of SLES: 51.9 g
add the SLES to the mixture with stirring using a frame paddle at 150 rpm, and leave to stir for 5 minutes.
   3/ Structuring of the Mixture
with gentle stirring (50 rpm), add 50% sodium hydroxide to reach pH 6.6. leave to homogenize thoroughly between each addition of sodium hydroxide.
   4/ Addition of the Amphoteric Surfactant
weigh out the mass of CAPB: 6.7 g
add the CAPB to the phase with stirring using a frame paddle at 50 rpm, and stir for the time necessary for homogenization.

B. Preperation Phase 2

1/ Dissolution of the Polymer in Water in a Beaker:

weigh out on a 0.001 g precision balance the mass of polymer A: 0.96 g
weigh out and add about 10 g of distilled water stir with a magnetic bar until the polymer has dissolved in the water.
   2/ Addition of Salt
weigh out on a 0.001 g precision balance the mass of salt: 0.10 g
dissolution in the mixture mix for 15 minutes using a magnetic bar
   3/ Adjustment of the pH
add 50% sodium hydroxide dropwise to reach the pH region 5.5-6.5. leave to stir for 5 minutes.
   4/ Addition of the Silicone Emulsion to the Mixture
weigh out on a 0.001 g precision balance the mass of silicone emulsion by weight: 1.54 g
   stir with a magnetic bar until homogeneous.

C. Addition of Phase 2 to Phase 1

With gentle stirring using a frame paddle (50 rpm), slowly add phase 2 to the water-SLES-CAPB-Carbopol Aqua SF1 mixture.

Leave to stir until homogeneous.

D. Addition of the Preserving Agent
Incorporate 2 drops of Kathon CG mix with a frame paddle at 150 rpm for 15 minutes.

E. Ajustment to pH 5.6

F. Addition of Water to Make 100 g

Characterization of the Formulation:

pH: 5.6
viscosity on Brookfield DV-I+ viscometer, T° C.=21° C., spindle 4, RV series, speed 10 rpm:
   measurement after 1 minute: 3860 mPa·s
Satisfactory stability

1-8. (canceled)

9. A cosmetic composition comprising:
(i) at least 0.05% by weight of an ampholytic copolymer comprising:
0.1% to 50% by number of cationic units (c) derived from the polymerization of at least one monomeric compound (c) of general formula I: 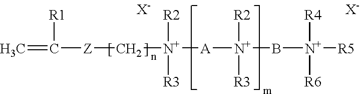
in which:
R₁ is a hydrogen atom or a methyl or ethyl group;
R₂, R₃, R₄, R₅ and R₆, which are identical or different, are linear or branched C₁-C₆ and optionally C₁-C₄ alkyl, hydroxyalkyl or aminoalkyl groups,
m is an integer from 0 to 10 and optionally from 0 to 2;
n is an integer from 1 to 6 and optionally from 2 to 4;
Z represents a —C(O)O— or —C(O)NH— group or an oxygen atom;
A represents a group (CH₂)ₚ, p being an integer from 1 to 6 and optionally from 2 to 4;
B represents a linear or branched C₂-C₁₂, optionally interrupted with one or more heteroatoms or hetero groups, and optionally substituted with one or more hydroxyl or amino groups; and
X⁻, which are identical or different, represent counterions;
anionic or potentially anionic units (a) derived from the polymerization of at least one hydrophilic monomer (a) bearing a function of acidic nature that is copolymerizable with (a), which is anionic or potentially anionic,
optionally units (n) derived from at least one ethylenically unsaturated monomer (n) of neutral charge, which is copolymerizable with (c) and (a), optionally an ethylenically unsaturated hydrophilic monomeric compound of neutral charge bearing one or more hydrophilic groups, which is copolymerizable with (c) and (a),
the amount of units (a) and optionally (n) being from 50% to 99.9% by number,
(ii) at least 0.1% by weight of an additive being:
ii.1: C₁₀-C₃₀ and preferably C₁₆-C₂₂ fatty acid esters of polyols or monoalcohols, or C₁₀-C₃₀ and preferably C₁₆-C₂₂ fatty alkyl ethers,
ii.2: synthetic polymers other than the copolymer (a), or
ii.3: polymers of natural origin and derivatives thereof,
(iii) optionally, at least one agent being
iii.1: polyorganosiloxanes, or
iii.2: oils other than polyorganosiloxanes,
(iv) optionally at least one surfactant,
(v) a cosmetically acceptable vector, optionally water.

10. The composition as claimed in the preceding claim, wherein the pH of the composition is between 3.5 and less than 5.5.

11. The composition as claimed in claim 9, wherein the pH of the composition is between 5.5 and 12, optionallt between 5.5 and 7.5.

12. The composition as claimed in claim 9, having:
from 0.1% to 0.5% by weight of the copolymer (i), and
from 0.15% to 7% by weight of the additive (ii).

13. The composition as claimed in claim 9, having from 0.1% to 5% by weight of a polyorganosiloxane agent (iii).

14. The composition as claimed in claim 9, having from 0.1% to 15% of an oil other than polyorganosiloxanes.

15. The composition as claimed in claim 9, comprising a conditioning agent being one of the agents (iii), and wherein the copolymer (i) and the conditioning agent are added into the composition in the form of a fluid concentrated ingredient, said composition having a total weight amount of copolymer (i) and of conditioning agent in the ingredient of at least 10% by weight, and optionally at least 60% by weight.

16. The composition as claimed in claim 9, wherein the ingredients (i), (ii), optionally (iii) and optionally (iv) are added into the composition separately.
